(19) United States
(12) Patent Application Publication
(10) Pub. No.: US 2025/0305047 A1
(43) Pub. Date: Oct. 2, 2025

(54) SINGLE CELL CO-SEQUENCING OF DNA METHYLATION AND RNA

(71) Applicant: The Regents of the University of California, Oakland, CA (US)

(72) Inventors: Huy Lam, La Jolla, CA (US); Andrew Richards, La Jolla, CA (US); Kun Zhang, La Jolla, CA (US)

(21) Appl. No.: 18/873,266

(22) PCT Filed: Jun. 9, 2023
(86) PCT No.: PCT/US2023/024930

§ 371 Date: Dec. 9, 2024

Related U.S. Application Data
(60) Provisional application No. 63/350,603, filed on Jun. 9, 2022.

(51) Int. Cl. C12Q 1/6883; C12Q 1/6874

(52) U.S. Cl. CPC C12Q 1/6883; C12Q 1/6874; C12Q 2600/154

(57) ABSTRACT

Methods, compositions and systems for co-sequencing DNA methylation and RNA from the same cell are provided. Also provided herein are gel beads which allow for the compartmentation of single cell nuclei and allow for processing of the nucleic acids therein by addition of DNA barcodes to allow for combinatorial indexing (e.g., three-layer combinatorial indexing) of the nuclei, thereby allowing the parallel processing of single cells in a high throughput manner. The method, compositions, and systems provided herein are capable of providing single cell sequencing data from tens of thousands or more cells in a single parallel experiment.

### CROSS REFERENCE

This application claims the benefit of U.S. Provisional Application No. 63/350,603 filed Jun. 9, 2022, which application is incorporated herein by reference in its entirety.

### GOVERNMENT SPONSORSHIP

This invention was made with government support under grant BNG7787 awarded by the National Institutes of Health. The government has certain rights in the invention.

### BACKGROUND

Cytosine-guanine dinucleotide (CpG) and non-CG DNA methylation have been associated with a variety of mammalian processes such as development, aging, and are disrupted in diseases such as cancer. Recent studies have shown that these methylation marks are cell-type specific and positively or negatively affect transcription factor binding affinity at regulatory elements such as enhancers and promoters (Mulqueen et al. 2018; Callaway et al. 2021). Single cell bisulfite sequencing opens the door for cell type specific methylome profiling for human cell atlas initiatives, identify cell-specific methylation markers associated with disease states, and provide additional epigenetic context to single cell RNA sequencing datasets. There exists a need for improved methods of performing single-cell sequencing analysis, particularly in a high throughput manner, and for performing DNA methylation analysis and RNA analysis in the same cell.

### SUMMARY OF THE INVENTION

The disclosure provides a single cell sequencing method that can sequence DNA methylation and RNA from the same cell at the scale of 50,000-100,000 cells, or more, using three 96 well plates.

In embodiments, this invention provides co-sequencing of DNA methylation and RNA from the same cell at this scale. Existing art with the same DNA methylation and RNA modality can only sequence tens of single cells. The technique described utilizes a combinatorial indexing concept to increase the cell throughput which has been described in previous art. However, a key innovation is the encapsulation of single cells with lysis buffer and acrylamide monomer in an oil emulsion using a microfluidic device droplet maker. The encapsulated cells are lysed and the acrylamide polymerized into a hydrogel. The encapsulated cells in hydrogel beads then undergo combinatorial indexing and novel library construction chemistries for DNA methylation and RNA sequencing. The approach provided herein describes the first method that involves the encapsulation of single cells or nuclei in hydrogel beads with the associated chemistries. In some instances, similar reactions were previously known in the art, but have been modified to be compatible with a gel bead platform as described herein.

In an aspect described herein is a method of parallel single-cell sequencing, comprising a) providing a plurality of cell nuclei or lysate thereof encapsulated in gel beads; b) performing reverse transcription within the gel beads to form complementary DNA (cDNA); c) partitioning the gel beads to a first plurality of vessels and adding a first DNA barcode to the cDNA and genomic DNA within the gel beads, each of the vessels of the first plurality of vessels having a unique first DNA barcode sequence d) pooling and re-partitioning the gel beads to a second plurality of vessels and adding a second DNA barcode to the cDNA and genomic DNA within the gel beads, each of the vessels of the second plurality of vessels having a unique second DNA barcode sequence; e) pooling and performing a second re-partitioning of the gel beads to a third plurality of vessels; f) pooling and performing a second re-partitioning of the gel beads to a third plurality of vessels; g) adding a third DNA barcode to the separated cDNA; h) performing bisulfite conversion of the separated genomic DNA and adding a third DNA barcode to the separated genomic DNA, wherein the third DNA barcode sequence is the same for genomic DNA and cDNA derived from the same cell nucleus; and i) sequencing the cDNA and the genomic DNA.

In embodiments, individual gel beads comprise a single cell nucleus or lysate thereof. In embodiments, providing the plurality of cell nuclei or lysate thereof encapsulated in gel beads comprises encapsulating the cell nuclei with a lysis buffer within a polymer matrix, wherein the polymer matrix forms the gel beads. In embodiments, providing the plurality of cell nuclei or lysate thereof encapsulated in gel beads comprises encapsulating the cell nuclei with a lysis buffer within a polymer matrix, wherein the polymer matrix forms the gel beads. In embodiments, the gel beads are comprised of an acrylamide polymer. In embodiments, the acrylamide polymer is prepared from acrylamide and bis-acrylamide in a ratio of about 100:1 (w/w). In embodiments, the gel beads have an average diameter of from about 100 to about 150 microns.

In embodiments, the gel beads comprise mRNA capture probes covalently attached to the gel beads. In embodiments, the mRNA capture probes act as reverse transcription primers during the reverse transcription step.

In embodiments, adding the first DNA barcode to the cDNA and the genomic DNA comprises transposon barcoding. In embodiments, the transposon barcoding is performed with transposon Tn5. In embodiments, the transposon barcoding is performed with transposon Tn5. In embodiments, the second DNA barcode is added to the cDNA and the genomic DNA by ligation. In embodiments, the ligation is performed with a T7 ligase.

In embodiments, the method further comprises amplifying the cDNA within the gel beads within the third plurality of vessels. In embodiments, separating the cDNA from the genomic DNA comprises centrifuging the gel beads to form a pellet and removing supernatant containing the cDNA. In embodiments, the third DNA barcode is added to the cDNA by polymerase chain reaction (PCR) of the cDNA in the supernatant. In embodiments, the performing bisulfite conversion of the separated genomic DNA comprises adding bisulfite conversion reagents to the pellet. In embodiments, the third DNA barcode is added to the genomic DNA by PCR of the genomic DNA. In embodiments, the method further comprises a gap filling step of amplifying the nucleic acids in the presence of a 5-methylcytosine dNTP.

In embodiments, the method obtains single cell sequencing data from at least 10,000 cell nuclei. In embodiments, the method obtains single cell sequencing data from at least 100,000 cell nuclei. In embodiments, each of the first, second, and third plurality of vessels comprises at least 96 individual vessels. In embodiments, each individual vessel of the first plurality of vessels comprises at least 200 gel beads containing a cell nucleus.

### BRIEF DESCRIPTION OF THE DRAWINGS

FIG. 1 shows a single cell sequencing process overview with three level combinatorial indexing as described herein.

FIG. 2A illustrates a process of preparing cDNA derived from a single nuclei within a gel bead according to an embodiment provided herein.

FIG. 2B illustrates the effect of different bis-acrylamide crosslinker levels on gel bead performance in library preparation (indicated as % C (percent crosslinker in the polymer, w/w)).

FIG. 3 shows a covalent capture strategy for retaining cDNA within gel beads according to an embodiment described herein.

FIG. 4 shows quantification of human and mouse reads for barcodes of both DNA and cDNA libraries in an indexing experiment performed using a covalent cDNA bead attachment strategy.

FIG. 5 shows graphical depictions of whole genome bisulfite sequencing construction methods.

FIG. 6 shows a depiction of a cDNA library prepared according to the embodiments provided herein before bisulfite conversion.

FIG. 7 shows a gap filling and linear amplification scheme after post bisulfite conversion according to an embodiments provided herein.

FIG. 8 shows library complexity analysis of single cell WGBS kidney libraries. Dotted lines indicate read cut-offs separating empty barcodes from occupied ones.

FIG. 9 shows a 3-Level sci-ATAC Combinatorial indexing scheme.

FIG. 10 shows Successful WGBS library construction with 3-level sci-ATAC design adapted to the WGBS protocol.

FIG. 11 shows Preliminary sequencing statistics of 3-level WGBS library construction method.

FIG. 12 shows an encapsulation and synthesis of full-length cDNA and subsequent digestion of RNA with RNAseH according to a protocol remove a TSO adapter sequences according to an embodiment herein.

FIG. 13 depicts a template switch oligonucleotide based combinatorial indexing method integrated with a WGBS 3-level indexing protocol as described herein.

FIG. 14 shows an approach to generating full-length cDNA with a gel bead as provided herein.

FIG. 15 shows a barcode collision rate assessment of in-gel cDNA synthesis for a single cell encapsulation approach as provided herein.

FIG. 16 shows Log normalized counts per million of the U87 in-tube and HCT116 encapsulated sample plotted (top). Log normalized counts per million of the HCT116 in-tube and HCT116 encapsulated sample plotted (bottom). The labeling of genes follows the convention: <Cell type>:<Marker Gene>. MALAT 1 was used as a marker gene and was detected in all libraries at high levels.

FIG. 17 shows an encapsulation strategy adapted from BAG-seq where the polymerization initiator, APS, is mixed with the polymerization precursors.

FIG. 18 shows an encapsulation strategy with polymer precursors separated from photoinitiator ammonium persulfate (APS).

FIG. 19 shows consistently low collision rates across two cell-line mixture encapsulation experiments.

FIG. 20 shows consistently low collision rates across two PBMC cell mixture encapsulation experiments.

FIG. 21 shows that optimization of both the DNA and cDNA libraries as provide herein results in 100× increases in library complexity.

FIG. 22 shows a primary analysis pipeline of a bioinformatics methods described herein.

FIG. 23 shows the database structure of libraries used to create sequencing statistic plots as described herein.

### DETAILED DESCRIPTION

### Certain Definitions

Various further aspects and embodiments of the disclosure are provided by the following description. Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and CC. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. F. Coligan et al, eds., 1991); Short Protocols in Molecular Biology (Wiley and Sons, 1999); Immunobiology (CA. Janeway and P. Travers, 1997); Antibodies (P. Finch, 1997). Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a nucleic acid sequence, a pharmaceutical composition, and/or a method that “comprises” a list of elements (e.g., components, features, or steps) is not necessarily limited to only those elements (or components or steps), but may include other elements (or components or steps) not expressly listed or inherent to the a nucleic acid sequence, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”. It is understood that aspects and embodiments of the invention described herein include “consisting” and/or “consisting essentially of” aspects and embodiments.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

“Amplification” refers to any known procedure for obtaining multiple copies of a target nucleic acid or its complement, or fragments thereof. The multiple copies may be referred to as amplicons or amplification products. Amplification, in the context of fragments, refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), reverse transcription polymerase chain reaction (RT-PCR), ligase chain reaction (LCR), strand-displacement amplification (SDA), and transcription-mediated or transcription-associated amplification. Amplification is not limited to the strict duplication of the starting molecule. For example, the generation of multiple cDNA molecules from RNA in a sample using reverse transcription (RT)-PCR is a form of amplification. Furthermore, the generation of multiple RNA molecules from a single DNA molecule during the process of transcription is also a form of amplification. During amplification, the amplified products can be labeled using, for example, labeled primers or by incorporating labeled nucleotides.

“Amplicon” or “amplification product” refers to the nucleic acid molecule generated during an amplification procedure that is complementary or homologous to a target nucleic acid or a region thereof. Amplicons can be double stranded or single stranded and can include DNA, RNA or both. Methods for generating amplicons are known to those skilled in the art.

“Codon” refers to a sequence of three nucleotides that together form a unit of genetic code in a nucleic acid.

“Codon of interest” refers to a specific codon in a target nucleic acid that has diagnostic or therapeutic significance (e.g. an allele associated with viral genotype/subtype or drug resistance).

“Complementary” or “complement thereof” means that a contiguous nucleic acid base sequence is capable of hybridizing to another base sequence by standard base pairing (hydrogen bonding) between a series of complementary bases. Complementary sequences may be completely complementary (i.e. no mismatches in the nucleic acid duplex) at each position in an oligomer sequence relative to its target sequence by using standard base pairing (e.g., G:C, A:T or A:U pairing) or sequences may contain one or more positions that are not complementary by base pairing (e.g., there exists at least one mismatch or unmatched base in the nucleic acid duplex), but such sequences are sufficiently complementary because the entire oligomer sequence is capable of specifically hybridizing with its target sequence in appropriate hybridization conditions (i.e. partially complementary). Contiguous bases in an oligomer are typically at least 80%, preferably at least 90%, and more preferably completely complementary to the intended target sequence.

“Configured to” or “designed to” denotes an actual arrangement of a nucleic acid sequence configuration of a referenced oligonucleotide. For example, a primer that is configured to generate a specified amplicon from a target nucleic acid has a nucleic acid sequence that hybridizes to the target nucleic acid or a region thereof and can be used in an amplification reaction to generate the amplicon. Also as an example, an oligonucleotide that is configured to specifically hybridize to a target nucleic acid or a region thereof has a nucleic acid sequence that specifically hybridizes to the referenced sequence under stringent hybridization conditions.

“Downstream” means further along a nucleic acid sequence in the direction of sequence transcription or read out.

“Upstream” means further along a nucleic acid sequence in the direction opposite to the direction of sequence transcription or read out.

“Polymerase chain reaction” (PCR) generally refers to a process that uses multiple cycles of nucleic acid denaturation, annealing of primer pairs to opposite strands (forward and reverse), and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. There are many permutations of PCR known to those of ordinary skill in the art.

“Position” refers to a particular amino acid or amino acids in a nucleic acid sequence.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

“DNA-dependent DNA polymerase” is an enzyme that synthesizes a complementary DNA copy from a DNA template. Examples are DNA polymerase I from E. coli, bacteriophage T7 DNA polymerase, or DNA polymerases from bacteriophages T4, Phi-29, M2, or T5. DNA-dependent DNA polymerases may be the naturally occurring enzymes isolated from bacteria or bacteriophages or expressed recombinantly, or may be modified or “evolved” forms which have been engineered to possess certain desirable characteristics, e.g., thermostability, or the ability to recognize or synthesize a DNA strand from various modified templates. All known DNA-dependent DNA polymerases require a complementary primer to initiate synthesis. It is known that under suitable conditions a DVA-dependent DNA polymerase may synthesize a complementary DNA copy from an RNA template. RNA-dependent DNA polymerases typically also have DNA-dependent DNA polymerase activity.

“DNA-dependent RNA polymerase” or “transcriptase” is an enzyme that synthesizes multiple RNA copies from a double-stranded or partially double-stranded DNA molecule having a promoter sequence that is usually double-stranded. The RNA molecules (“transcripts”) are synthesized in the 5′-to-3′ direction beginning at a specific position just downstream of the promoter. Examples of transcriptases are the DNA-dependent RNA polymerase from E. coli and bacteriophages T7, T3, and SP6.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) ““Gapped BLAST and PSI-BLAST: a new generation of protein database search programs” Nucleic Acids Res. 25:3389-3402, and Zhang et al (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

A “label” refers to a moiety attached (covalently or non-covalently), or capable of being attached, to a molecule, which moiety provides or is capable of providing information about the molecule (e.g., descriptive, identifying, etc. information about the molecule) or another molecule with which the labeled molecule interacts (e.g., hybridizes, etc.). Exemplary labels include fluorescent labels (including, e.g., quenchers or absorbers), weakly fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like.

A “linker” refers to a chemical moiety that covalently or non-covalently attaches a compound or substituent group to another moiety, e.g., a nucleic acid, an oligonucleotide probe, a primer nucleic acid, an amplicon, a solid support, or the like. For example, linkers are optionally used to attach oligonucleotide probes to a solid support (e.g., in a linear or other logic probe array). To further illustrate, a linker optionally attaches a label (e.g., a fluorescent dye, a radioisotope, etc.) to an oligonucleotide probe, a primer nucleic acid, or the like. Linkers are typically at least bifunctional chemical moieties and in certain embodiments, they comprise cleavable attachments, which can be cleaved by, e.g., heat, an enzyme, a chemical agent, electromagnetic radiation, etc. to release materials or compounds from, e.g., a solid support. A careful choice of linker allows cleavage to be performed under appropriate conditions compatible with the stability of the compound and assay method. Generally a linker has no specific biological activity other than to, e.g., join chemical species together or to preserve some minimum distance or other spatial relationship between such species. However, the constituents of a linker may be selected to influence some property of the linked chemical species such as three-dimensional conformation, net charge, hydrophobicity, etc. Exemplary linkers include, e.g., oligopeptides, oligonucleotides, oligopolyamides, oligoethyleneglycerols, oligoacrylamides, alkyl chains, or the like. Additional description of linker molecules is provided in, e.g., Hermanson, Bioconjugate Techniques, Elsevier Science (1996), Lyttle et al. (1996) Nucleic Acids Res. 24 (14): 2793, Shchepino et al. (2001) Nucleosides, Nucleotides, & Nucleic Acids 20:369, Doronina et al (2001) Nucleosides, Nucleotides, & Nucleic Acids 20:1007, Trawick et al. (2001) Bioconjugate Chem. 12:900, Olejnik et al. (1998) Methods in Enzymology 291:135, and Pljevaljcic et al. (2003) J. Am. Chem. Soc. 125 (12): 3486, all of which are incorporated by reference.

“Fragment” refers to a piece of contiguous nucleic acid that contains fewer nucleotides than the complete nucleic acid.

“Hybridization,” “annealing,” “selectively bind,” or “selective binding” refers to the base-pairing interaction of one nucleic acid with another nucleic acid (typically an antiparallel nucleic acid) that results in formation of a duplex or other higher-ordered structure (i.e. a hybridization complex). The primary interaction between the antiparallel nucleic acid molecules is typically base specific, e.g., A/T and G/C. It is not a requirement that two nucleic acids have 100% complementarity over their full length to achieve hybridization. Nucleic acids hybridize due to a variety of well characterized physio-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, New York), as well as in Ausubel (Ed.) Current Protocols in Molecular Biology, Volumes I, II, and III, 1997, which is incorporated by reference.

The term “attached” or “conjugated” refers to interactions and/or states in which material or compounds are connected or otherwise joined with one another. These interactions and/or states are typically produced by, e.g., covalent bonding, ionic bonding, chemisorption, physisorption, and combinations thereof.

“Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

### Single Cell DNA Methylation Sequencing Techniques

Single cell DNA methylation can be assayed using whole genome-bisulfite sequencing (WGBS) or reduced representation bisulfite sequencing (RRBS). WGBS interrogates the DNA methylation status of the whole genome. Most single cell WGBS studies have focused on mammalian brain or stem cell tissues (Argelaguet et al. 2019; Angermueller et al. 2016; Luo et al. 2018). Compared to other tissues, these tissues exhibit elevated non-CG methylation which greatly assists in the clustering of single cells. In contrast, the low level of non-CG methylation requires the use of CG methylation to cluster single cells. In single cell WGBS analyses of kidney tissue, it has been observed that the number of non-CG cytosine sites far exceeds the number of CG sites. Thus, the vastly lower number of potentially differentially methylated cytosine positions lowers the ability to cluster single cells in these tissues.

To cluster cells, WGBS typically requires a high sequencing depth of at least 1 million unique reads per cell. RRBS aims to lower these sequencing costs by enriching for CG sites by using a restriction enzyme, MspI, that cuts at high density CG islands. However, RRBS does not recover biologically relevant non-CpG methylation and misses low density CG sites. Thus, single cell RRBS technologies still require sequencing depths in the millions to reads like WGBS to perform downstream analyses (Gu et al. 2021; Hu et al. 2016). In addition, RRBS does not recover variable cell type specific non-CG methylation as found in the context of brain and stem cell tissues which limits its use as a platform technique.

Typically, thousands of cell libraries are needed to characterize heterogenous human tissues. snmC-seq is by a large margin the most prolific single cell WGBS method and has been used as the backbone to methylome cell atlas studies with the ability to generate thousands of single cell methylomes per study, 10-fold higher than most other techniques (Callaway et al. 2021). Briefly, extracted nuclei are sorted into individual reaction vessels which are given a well-specific DNA barcode during library construction (Callaway et al. 2021; Mulqueen et al. 2018). Using a liquid handling system, this protocol can reportedly generate an astonishing 10,000 single cell methylomes per week by automating the thousands of reactions in parallel (Luo et al. 2022). The optimized adaptation of this protocol in 384 well plates to liquid handlers is key to the high throughput of this method. However, the use of liquid handlers prevents the practical widespread adoption of this method and its ability to practically scale to millions of cells like other single cell technologies (Cao et al. 2020; Domcke, Hill, Daza, Cao, O'Day, Pliner, Aldinger, Pokholok, Zhang, Milbank, Zager, Glass, Steemers, Doherty, Trapnel, et al. 2020).

Recent combinatorial indexing methods offer a potential solution to exponentially scale the cell throughput of single cell sequencing technologies without the extensive use of liquid handlers. Briefly, these technologies leverage a split-pool barcoding scheme that virtually creates an exponentially scaled barcode space. For example, a barcode space of 56 million barcodes can be created with 3-levels of combinatorial barcoding using 3×384 well plates. The single cell input into this barcode space is typically restricted to 10% of this barcode space to minimize the chance that two cells have the same barcode. This technique can potentially sequence millions of cells and has been demonstrated to perform single cell RNA and chromatin accessibility sequencing of organ systems (Cao et al. 2020; Domcke, Hill, Daza, Cao, O'Day, Pliner, Aldinger, Pokholok, Zhang, Milbank, Zager, Glass, Steemers, Doherty, Trapnell, et al. 2020). sci-MET is a recently published single cell WGBS technique that uses a 2-level combinatorial indexing approach. Isolated nuclei are first fixed with formaldehyde and then nucleosome depleted whereby a careful balance is struck between the denaturation of chromatin organization proteins for whole genome coverage and structural integrity of the nucleus. Next, thousands of nuclei per well are flow sorted into a 96 well plate, and a well specific DNA barcode is inserted using Tn5 transposase into all genomic fragments. The nuclei are then mixed and then roughly 10 nuclei are flow sorted into a second 96 well plate where bisulfite conversion takes place. Post bisulfite conversion, a second well-specific barcode is added during the final PCR. Using 2×96 well plates, this protocol demonstrated the ability to generate roughly 1000 single cells per experiment at a mean sequencing depth of 200,000 reads per cell. As indicated in this study, this method has at least 5-fold lower library complexity compared to snmC-seq (Mulqueen et al. 2018). Because the extent of DNA accessibility to Tn5 barcoding is in tension with the structural integrity of the nucleus, the low coverage may be due to continued existence of DNA binding proteins after nucleosome depletion. SnmC-seq still comfortably scales to the throughput of sci-MET. However, continued innovation in combinatorial indexing schemes such as the addition of a third level of indexing would out scale snmC-seq as barcode layer exponentially scales the cell throughput. In addition to technological challenges to generate single cell WGBS datasets, the biological interpretation of WGBS data is also an immense challenge.

### Challenges in Biological Interpretation of Single Cell DNA Methylation Data Sets

CG methylation is typically associated with gene repression. For example, X-chromosome inactivation is a critical feature of female mammalian embryonic development which is established and maintained by CG methylation gene repression (Heard, Clerc, and Avner 1997). SnmCAT-seq, derived from snmC-seq, was recently developed to profile the transcriptome, DNA cytosine methylation, and chromatin accessibility in postmortem human frontal cortex tissue (Luo et al. 2022). Currently, this is the only study that has generated thousands of single cell coupled WGBS and RNA datasets as single cell per well methods can only reasonably generate low hundreds of cells without liquid handler robotics. This study showed that CH methylation within gene bodies of neuronal cells can have different effects in different contexts. For example, the expression of KCNIP4 has a strong negative correlation between RNA expression and gene body methylation in excitatory neurons but a slight positive correlation in in inhibitory neurons. In contrast, the expression of ADARB2 shows a strong negative correlation with gene body methylation in inhibitory neurons but a slight positive correlation in excitatory neurons. Interestingly, the expression of GPC5 is positively associated with gene body methylation for both inhibitory and excitatory neurons (Luo et al. 2022). Another noteworthy co-sequencing method called scNMT-seq has been used to profile the transcriptome and methylome of differentiating stem cells (Clark et al. 2018; Argelaguet et al. 2019; Angermueller et al. 2016). An RNA expression predictive model using WGBS based on these scNMT-seq studies found positive correlations between DNA methylation at promoters and gene expression for those genes. This correlation is opposite from most bulk DNA methylation studies Because the data used for training this model is from stem cell rich tissue, this opposite correlation could be a distinguishing feature of stem cells (Uzun, Wu, and Tan 2021). Therefore, the modulation of gene activity of a nearby methylated feature is extensively cell type dependent.

The high context dependency of a methylation mark's effect on nearby gene expression presents a formidable challenge in integrating single cell methylation with the more broadly used single cell RNA modality. In contrast, single cell DNA accessibility shows relatively consistent positive correlations with nearby RNA gene expression. The Cicero computational method demonstrated that this general positive correlation can be used to quantify the relative predicted gene expression of a particular gene using only the number of DNA accessible sites and their distances from that gene (Pliner et al. 2018). Since DNA methylation can have an ambivalent effect or no effect on gene expression depending on the context, computational methods have limited ability to couple proximal methylated features to a gene without additional RNA information. Nevertheless, single cell WGBS in the form of snmC-seq and snmCAT-seq has demonstrated cell-type clustering of brain cells with similar resolution to RNA (Callaway et al. 2021; Luo et al. 2022). In contrast, single cell DNA accessibility clustering of human brain cells have been shown to be lowest in resolution (Chen, Lake, and Zhang 2019; Lake et al. 2018). Thus, the integration of the methylome and transcriptome could potentially reveal how DNA methylation, at loci resolution, establishes and maintains specific cell type identity in the broader context of DNA methylation associated phenomena such as cancer and aging.

Multi-omic methods such as snmCAT-seq and scNMT-seq are therefore critical to elucidate the epigenetic context of DNA methylation for a specific cell type. These methods integrate the RNA expression and the whole genome DNA cytosine methylation of the same single cell. Nuclei are first isolated from brain tissue followed by the methylation of cytosines in the GC context of DNA accessible cytosines with GpC methyltransferase. DNA binding proteins such as nucleosomes block the inaccessible GC positions from receiving the methyl groups. During bisulfite sequencing, the unmethylated cytosines convert to thymines. As a result, cytosine conversions in the GC context are interpreted as inaccessible and vice versa. The nuclei are then flow sorted into individual reaction wells where reverse transcription and cDNA amplification with methylated cytosine takes place using the SMART-Seq protocol. The reaction then undergoes bisulfite conversion follow by post bisulfite adapter ligation using the adaptase enzyme. DNA and cDNA libraries are then co-sequenced and bioinformatically split based on highly methylated and lowly methylated reads in the CH sequence motif. Highly methylated reads are presumed to be cDNA reads which were amplified with methylated cytosine prior to bisulfite conversion while DNA reads are lowly methylated, as expected for human cells. This crucially allows for the hypothesized biological relevance of a particular methylated locus to be cross-validated with the RNA expression of nearby genes. Like snmC-seq, this method achieves high cell throughput by flow sorting nuclei into individual wells in a 384 well plate and using optimized liquid handlers. Without one, a team would have to run the snmCAT-seq protocol in at least 5,000 individual wells to generate the roughly 4,358 single nuclei datasets reported.

### Challenges in Single Cell Clustering of Terminally Differentiated Cell-Types

All single cell methylation technologies suffer from low coverage of the genome. In the mammalian diploid genome, there are only two copies of a cytosine site that can be detected. In contrast, the RNA transcript drop-outs during library preparation is mitigated by the naturally high copy numbers of gene transcripts. In addition, the harsh bisulfite chemistry required for DNA methylome sequencing causes extensive DNA loss due to double stranded breaks. Thus, most genomic fragments are lost during WGBS library preparation. For example, the maximum genome coverage possible projected library complexity of scnmC-seq is 30% per cell. This poses a significant challenge to computationally cluster single cells into cell-types. In summary, the methylated cytosine information is binned across vast genomic windows (typically 100 kb in size) by cell. Only bins with high coverage across all cells are considered. Single cells of the same cell type can be clustered based on similar methylation levels across these bins. Generally, millions of reads per cell are minimally required to capture enough shared methylated cytosine sites across the bins for clustering. For example, the average sequencing depth of scnmC-Seq is 5 million reads per cell to cover approximately 10% of the genome per cell to cluster brain cells (Callaway et al. 2021) Notably, most single cell methylation or methylation and RNA co-sequencing studies focus on mammalian neuronal or stem cell populations where cell type specific CH methylation is elevated. For example, neurons can have 5-fold more CH methylation compared to glia. In addition, both tissue types contain cell-type specific elevated 5-hydroxymethylcytosine which is captured, but not distinguished from, methylcytosine during WGBS. For example, it's estimated that 5 hmC methylation is 40% as abundant as CG methylation in Purkinje cells (Kriaucionis and Heintz, n.d.). Therefore, informative methylation features are bolstered by the additional cell type specific 5 hmC sites. In contrast, terminally differentiated tissues demonstrate low levels of CH methylation. Thus, CG methylation would be used to cluster single cells. It has been found that the number of CH sites can be over 5-10 fold more abundant than CG sites based on our WGBS study on kidney tissue. Therefore, it's plausible that the required sequencing depth to cluster terminally differentiated cell types will require vastly more than 10% genome coverage, possibly beyond the snmC-seq projected maximum library complexity of 30% (Luo et al. 2018). Unsurprisingly, single cell methylation of terminally differentiated tissue remains vastly understudied because of these complications.

Multi-omic technologies such as snmCAT-seq offer part of the solution to studying the methylome of terminally differentiated tissues. With multi-omic RNA and WGBS co-sequencing, single cells can be clustered and grouped into a pseudo-bulk with as little as 50,000 unique RNA reads per cell. These cell type group labels can be then transferred to the WGBS library where these same cells can be pooled into a pseudo-bulk. Differential methylation analysis can then be performed between these pseudo-bulk profiles defined by the RNA cell type label. This framework leverages the powerful ability of single cell RNA-seq to discriminate most cell types as demonstrated by numerous cell atlas studies of human organs using the transcriptome (Quake 2022). For example, if the single cell methylome library is sequenced to 1,000,000 reads per cell, roughly 500 cells within a cell type pseudo-bulk would be needed to have 30× coverage of that cell type. This high coverage could plausibly contain enough CG methylation information to identify novel cell-type specific CG methylation features, currently understudied in terminally differentiated tissue. Furthermore, the methylome of rare cell types that can only be observed in high throughput single cell RNA-seq experiments could also be profiled. This analysis framework requires an ultra-high throughput method on the order of tens of thousands of cells. In essence, a higher throughput co-sequencing assay results in higher methylome coverage of a particular cell type as more cells constitute the corresponding methylome pseudo-bulk. All DNA methylation and RNA co-sequencing platforms currently lack the cell throughput required for this analysis.

### Exemplary Improvements Provided Herein

The embodiments provided and described herein build upon existing multi-omic DNA methylation and RNA co-sequencing technologies by expanding the throughput from hundreds of cells to tens of thousands of cells per experiment. In embodiments, described herein is an ultra-high cell throughput multi-omic DNA methylation and RNA co-sequencing platform as the basis for the pseudo-bulk analysis framework previously mentioned. In embodiments, the method utilizes a combinatorial indexing approach inspired by sci-MET, but crucially increases the throughput of this scheme 100-fold to allow sequencing of tens of thousands of cells using 3×96 well plates by adding a third round of barcoding in one experiment. Embodiments provided herein demonstrate how the nucleosome depletion process as described in sci-MET severely reduces the structural integrity of the nucleus, preventing the additional reverse transcription and barcoding reactions required for 3-level co-sequencing of DNA methylation and RNA. Also provided herein is a solution that involves the simultaneous encapsulation and lysis of single cells or nuclei within polyacrylamide hydrogel beads. This combinatorial indexing vessel, in contrast to nucleosome depleted nuclei, displays drastically higher vessel stability, allowing for the robust addition of reverse transcription and additional barcoding reactions beyond 3-levels. For example, the polyacrylamide remains intact after exposure to high concentrations of SDS and protease K which is crucial to robustly denature DNA binding proteins. In embodiments, provided herein, the method provides a 3×96 well plate that can sequence 50,000-100,000 single cells per experiment. In embodiments, it is expected that the methods provided herein could be readily adapted to a 3×384 well plate allowing for the sequencing of 3-5 million single cells per experiment. Ultimately, the embodiments described herein provide the next step in single cell WGBS and RNA co-sequencing technology development by unlocking the possibility to profile the methylomes of terminally differentiated tissues using an ultra-high throughput approach.

Embodiments provided herein describe the development of a novel combinatorial indexing method where single cells or nuclei are simultaneously encapsulated and lysed within polyacrylamide gel beads. In embodiments, these gel beads act as the vessel that compartmentalizes both the DNA and RNA during the barcoding steps. In embodiments, this gel bead encapsulation method provided advantages as compared to other methods which comprises adding additional reactions to reverse transcribe RNA and performing additional barcoding using nucleosome depleted nuclei. In embodiments, the design of this novel gel bead platform is provided, resulting in the development of a gDNA and RNA co-sequencing platform. In embodiments, the platform described herein can be used in the profiling of DNA copy number variations in various cancers and their effects on cancer cell RNA expression.

Also described herein is the design of a novel bisulfite sequencing method to solve the unique challenges involving the bisulfite conversion of these polyacrylamide gel beads. In embodiments, this novel approach could solve some existing problems in bulk WGBS. It is contemplated that the method provided herien can be readily slight optimized to offer a better bulk bisulfite sequencing method. Further described herein is a whole methylome and transcriptome co-sequencing method.

In embodiments, the methods provided herein provide an improved method of combinatorial indexing for large scale (e.g., high throughput) single-cell sequencing. Combinatorial indexing is a virtual single cell sequencing technique which allows high-throughput analysis of a large plurality of samples without the need for specifically generating a unique molecular barcode for each sample on an individual basis. In embodiments, combinatorial indexing comprises adding a first barcode sequence to a plurality of cellular DNA samples, then subsequent pooling and re-distributing the cellular DNA samples and adding subsequent barcodes in a manner such that it is a low probability that any two samples end up with the same combination of barcode sequences. In embodiments provided herein are three-level combinatorial indexing schemes (e.g., schemes which comprise separately adding three independent barcode sequences to a DNA sample such that there is a low probability that any two cellular samples comprise the same set of three barcodes).

Traditional combinatorial indexing schemes rely on the nucleus of a cell to act as a vessel that contains the DNA and RNA of the cells during the barcoding and shuffling process. However, with DNA methylation sequencing, most DNA is tightly wrapped around histone proteins, making it unavailable for bisulfite conversion and/or subsequent enzyme processing for adding the barcodes (e.g., partition specific barcodes). There exists a challenge that destruction of the histone proteins (necessary for the conversion and barcoding process) leads to destruction of the cell nucleus since they are made of the same basic amino acids, thereby causing leakage of the nucleic acids. Since combinatorial indexing requires an intact vessel during the barcoding process, this highly problematic.

Thus, in embodiments, the instant disclosure solves this problem by providing a gel bead with sufficient strength to withstand conditions able to unwrap (e.g., denature and/or destroy) histones to allow bisulfite conversion and enzymatic barcoding of the nucleic acids of the sample, yet possesses sufficient porosity or other factors (e.g., size) which allow the nucleic acids to be subsequently released in order to effectuate further processing of the nucleic acids for sequencing. In further embodiments, the disclosure described herein provides unique and optimized chemistries in order to effectuate the desired barcoding and/or other processing of nucleic acids (e.g., complementary DNA and/or genomic DNA) in order to allow for a three-level combinatorial indexing scheme to be successfully carried out in a manner which allows methylation sequencing of genomic DNA as well as RNA sequencing of the cells, thereby providing detailed information on a single-cell level of a large number of cells in parallel.

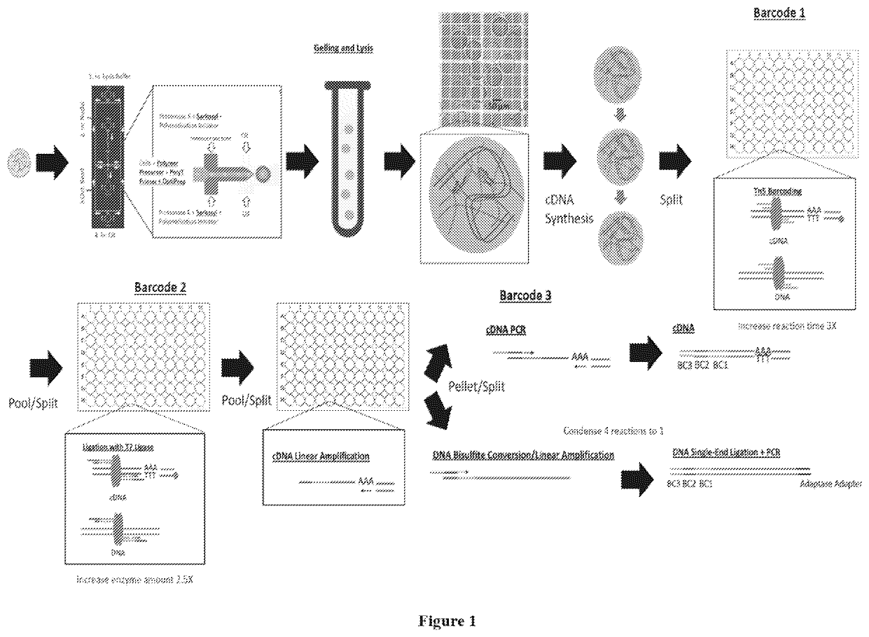

An exemplary overview of a parallel single cell sequencing workflow based on combinatorial indexing according to the instant disclosure is depicted in FIG. 1. Briefly, cell nuclei (or, in certain embodiments, whole cells) are encapsulated in a polymeric gel bead with a lysis buffer suitable for lysing the nucleus and genome packing proteins, thereby freeing the DNA therefrom. Upon removal from the device which encapsulates the nuclei and lysis buffer, the beads are allowed to gel. Preferably, the plurality of gel beads produced from the device include gel beads which contain single nuclei and few gel beads which contain multiple nuclei. The plurality of gel beads produced can include large numbers of gel beads which contain no nuclei (empty gel beads, e.g., more than 90% empty gel beads). After gelling of the beads, cDNA is synthesized from the RNA within the beads. The gel beads are then partitioned (e.g., to a 96-well plate) and a first DNA barcode specific to each vessel (e.g., each well of the 96-well plate) is added to the cDNA and genomic DNA (e.g., by a transposase barcoding method, such as one using Tn5). In embodiments, the gel beads are then pooled and re-partitioned (e.g., to a second 96-well plate) and a second DNA barcode added (e.g., by a ligation with T7 ligase) to the cDNA and genomic DNA, each second DNA barcode likewise being unique to each well. In embodiments, the gel beads are then pooled and re-partitioned again (e.g., to a third 96-well plate). In embodiments, from this third partition (e.g., the third 96-well plate), gel beads are pelleted (e.g., by centrifugation), thereby providing genomic DNA in the pellet and cDNA in the supernatant. In embodiments, the supernatant is removed and a third DNA barcode is added to the cDNA (e.g., by PCR). In embodiments, the genomic DNA in the pellet is then converted with bisulfite and linearly amplified, then subsequently barcoded (e.g., by PCR) with the third DNA barcode (each nucleic acid included in the same vessel (e.g., same well of the 96-well plate) receiving the same third DNA barcode which is unique to that vessel). In embodiments, the nucleic acids are then sequenced, thereby providing single-cell sequencing data for both RNA (as sequenced from the cDNA) and genomic DNA (e.g., methylation sequencing).

In an aspect provided herein is a method of single cell co-sequencing of DNA methylation and RNA of >100,000 cells with 3×96-well plates or 3×384-well plates. In embodiments, this is accomplished without the need for flow cytometry. In embodiments, the method comprises the use of encapsulated gel beads in a combinatorial indexing scheme. In embodiments, the method comprises reverse transcription which converts RNA to cDNA which can be barcoded and sequenced. In embodiments, the method comprises destruction of DNA organizing proteins (e.g., nucleosomes, histones, etc.). In embodiments, the method utilizes two barcoding reactions where the nucleic acids (DNA and cDNA) are compartmentalized in a vessel (e.g., a gel bead).

In embodiments, use of the gel beads as provided herein provides distinct advantages over other methods of single cell sequencing (e g., Sci-MET) In embodiments, barcoding reactions degrade the structural integrity of the nucleus, which causes problems in other published nucleosome depleted combinatorial indexing schemes, which are thereby limited to one barcoding reaction due to subsequent leaking of the nucleic acids. In embodiments, methods which utilize multiple barcoding steps which require buffer exchange (e.g., too remove excess enzyme from the previous reaction and add co-factors required for the next reaction). This is typically done by pelleting the nuclei with a centrifuge, removing the supernatant, and resuspending the nuclei in the reaction mix for the next reaction. Because nucleosome depleted nuclei are structurally compromised, nucleosome depleted protocols generally require a flow cytometry based (e.g., fluorescence activated cell sorting (FACS)) cell sorter to gently exchange the buffer (a huge machine cost). Thus, in embodiments, the use of gel beads as described herein provide advantages over other methods owing to the fact that the gel beads are engineered to a) destroy the nucleosomes (e.g., are stable enough to withstand lysis conditions which allow for denaturing of nucleosomes), b) possess a small enough pore size to immobilize nucleic acids within the bead for barcoding (e.g., by optimizing the polymer which makes up the gel bead), c) possess a large enough pore size such that diffusion of enzymes and DNA barcodes to barcode the nucleic acids can enter the gel bead, and d) be strong enough to withstand the barcoding reactions and other steps (e.g., centrifugation, washes, etc.) without the need for flow cytometry. Thus, in embodiments described herein, are gel beads which possess a desired pore size (e.g., owing to the ratio of copolymers (e.g., acrylamide and bis-acrylamide) used in their manufacture) and a desired bead radius (e.g., sufficiently large to allow the barcoding chemistry and other enzymatic reactions to occur). In embodiments, the gel beads provided herein allow for one or more of a) entrapment of DNA and RNA from single cells; b) first strand synthesis of cDNA (e.g., DNA converted from RNA) via in-bead reverse transcription, c) generation of second strand synthesis of cDNA, d) simultaneous in-bead first barcoding of cDNA and genomic DNA (e.g., via Tn5 tagmentation), and/or e) simultaneous in-bead second barcoding of cDNA and DNA (e.g., via a ligation reaction, such as that provided by commercial sources such as the snmCAT-seq by IDT Biologika). In embodiments, the gel beads further allow for an in-bead gap filling step with methylated cytosines to protect DNA barcodes from bisulfite conversion. In embodiments, the gel beads further allow for extraction of cDNA and bisulfite converted DNA after linear amplification with repeated pelleting and resuspension.

In an aspect, provided herein, is a method of parallel single-cell sequencing. In embodiments, the method comprises providing a plurality of cell nuclei or lysate thereof encapsulated in gel beads. In embodiments, the method comprises performing reverse transcription within the gel beads to form complementary DNA (cDNA). In embodiments, the method comprises partitioning the gel beads to a first plurality of vessels and adding a first DNA barcode to the cDNA and genomic DNA within the gel beads, each of the vessels of the first plurality of vessels having a unique first DNA barcode sequence. In embodiments, the method comprises pooling and re-partitioning the gel beads to a second plurality of vessels and adding a second DNA barcode to the cDNA and genomic DNA within the gel beads, each of the vessels of the second plurality of vessels having a unique second DNA barcode sequence. In embodiments, the method comprises pooling and performing a second re-partitioning of the gel beads to a third plurality of vessels. In embodiments, the method comprises separating the cDNA from the genomic DNA. In embodiments, the method comprises adding a third DNA barcode to the separated cDNA. In embodiments, the method comprises performing bisulfite conversion of the separated genomic DNA. In embodiments, the method comprises adding a third DNA barcode to the separated genomic DNA. In embodiments, the third DNA barcode sequence is the same for genomic DNA and cDNA derived from the same cell nucleus. In embodiments, the method comprises sequencing the cDNA and the genomic DNA. In embodiments, the steps are performed in the order in which they are provided supra.

In embodiments, the method comprises providing a plurality of cell nuclei or lysate thereof encapsulated in gel beads. In embodiments, individual gel beads (e.g., those of the plurality) comprise a single cell nucleus or lysate thereof. In embodiments, the method comprises providing a plurality of gel beads which comprise a single cell nucleus or lysate thereof (e.g., encapsulated therein). In embodiments, the plurality of gel beads which contain a single cell nucleus or lysate thereof can be among other gel beads of different compositions. For example, it is contemplated that a plurality of gel beads which comprise a single cell nuclease or lysate thereof can be interspersed with gel beads which comprise no cell nucleus or corresponding lysate, can be interspersed with gel beads which comprise multiple cell nuclei or lysates thereof, or a combination of both. Preferably, the plurality of cell nuclei or lysate thereof encapsulated within gel beads will be interspersed with only a minimal number of gel beads which comprise multiple nuclei or lysates thereof (e.g., within a population of gel beads, less than 1%, less than 0.5%, or less than 0.1% of the gel beads will comprise multiple nuclei).

In embodiments, the plurality of gel beads which contain a single cell nucleus or lysate thereof will be interspersed with a high number of gel beads which contain no cell nuclei or lysates thereof. In embodiments, such a configuration is preferable because it ensures that in filling the gel beads with cell nuclei, there are a minimal number of gel beads which comprise multiple cell nuclei or lysates thereof (e.g., but forming the encapsulations at a limiting dilution of the cell nuclei). In embodiments, the plurality of gel beads which comprises a single cell nuclei or lysate thereof will be interspersed with substantially more gel beads which contain no nuclei or lysates thereof (e.g., there will be an excess of “empty” gel beads of at least 2-fold, 3-fold, 4-fold, 5-fold, 6-fold, 7-fold, 8-fold, 9-fold, or 10-fold compared to gel beads which comprise a cell nucleus or lysate thereof). In embodiments, in a population of gel beads which includes the desired plurality of gel beads comprising a single cell nucleus or lysate thereof, the population will comprise at least 75%, at least 80%, at least 85%, or at least 90% of gel beads which contain no cell nucleus or lysate thereof.

In embodiments, the gel beads which contain a cell nucleus or lysate thereof can comprise other components (e.g., other parts of the cell or lysates thereof). In embodiments, the gel beads which contain a cell nucleus or lysate thereof comprise a whole cell or lysate thereof (e.g., the cell nuclei are not first isolated prior to encapsulation with lysis buffer).

In embodiments, providing the plurality of cell nuclei or lysate thereof encapsulated in gel beads comprises encapsulating the cell nuclei with lysis buffer within a polymer matrix. In embodiments, the polymer matrix forms the gel beads. In embodiments, providing the plurality of gel beads comprises mixing of multiple aqueous streams to provide the final contents of the gel bead. In embodiments, providing the mixing of multiple aqueous streams comprises mixing a first stream comprising the cell nuclei (e.g., as isolated cell nuclei or a whole cells) and polymer precursor(s) (e.g., acrylamide and/or bis-acrylamide) with a second stream which comprises the lysis buffer components (e.g., proteases and/or detergents) as well as a polymerization initiator. In embodiments, mixing of these aqueous streams forms a polymer matrix owing to activation of the polymerization initiator (e.g., ammonium persulfate). In embodiments, the polymer matrix hardens to form the gel bead (e.g., after a suitable period of time, such as at least 2, 4, 6, 8, 12, 16, or 24 hours).

In embodiments, the lysis buffer comprises reagents suitable for lysing the cell nucleus. In embodiments, the lysis buffer comprises one or more detergents, surfactants, salts, buffers, proteases, or other suitable components. In embodiments, the lysis buffer comprises a detergent. In embodiments, the lysis buffer comprises an ionic detergent, an non-ionic detergent, or a combination thereof. In embodiments, the lysis buffer comprises a protease. In embodiments, the lysis buffer comprises proteinase K. In embodiments, the lysis buffer comprises sarkosyl (sodium lauroyl sarcosinate).

In embodiments, the encapsulating comprises mixing the cell nuclei, the lysis buffer, and the polymer matrix within a water-in-oil droplet. In embodiments, the aqueous components of the gel bead are mixed and then entered into an oil stream in order to provide the water-in-oil droplet. Any suitable water immiscible oil can be used to form the water-in-oil droplet. In embodiments, the oil of the water in oil droplet is a hydrophobic material (e.g., a fluorinated oil). Exemplary compatible oils include those described in, for example, U.S. Pat. No. 10,105,703.

In embodiments, the gel beads are comprised of an acrylamide polymer. In embodiments, the gel beads are comprised of a mixture of polymerized acrylamide and bis-acrylamide. In embodiments, the acrylamide polymer is prepared from acrylamide and bis-acrylamide in a ratio of about 5:1 (w/w), about 10:1 (w/w), about 15:1 (w/w), about 20:1 (w/w), about 25:1 (w/w), about 30:1 (w/w), about 35:1 (w/w), about 40:1 (w/w), about 45:1 (w/w), about 50:1 (w/w), about 55:1 (w/w), about 60:1 (w/w), about 65:1 (w/w), about 70:1 (w/w), about 75:1 (w/w), about 80:1 (w/w), about 85:1 (w/w), about 90:1 (w/w), about 95:1 (w/w), about 100:1 (w/w), about 110:1 (w/w), about 120:1 (w/w), about 130:1 (w/w), about 140:1 (w/w), or about 150:1 (w/w). In embodiments, the acrylamide polymer is prepared from acrylamide and bis-acrylamide in a ratio of about 20:1 (w/w) to about 150:1 (w/w). In embodiments, the acrylamide polymer is prepared from acrylamide and bis-acrylamide in a ratio of about 100:1. In embodiments, the acrylamide polymer is prepared from acrylamide and bis-acrylamide in a ratio of about 50:1 (w/w) to about 200:1 (w/w). In embodiments, the acrylamide polymer is prepared from acrylamide and bis-acrylamide in a ratio of about 75:1 (w/w) to about 150:1 (w/w). In embodiments, the acrylamide polymer is prepared from acrylamide and bis-acrylamide in a ratio of about 80:1 (w/w) to about 120:1 (w/w). In embodiments, the acrylamide polymer is prepared from acrylamide and bis-acrylamide in a ratio of about 90:1 (w/w) to about 110:1 (w/w). In embodiments, the acrylamide polymer has a crosslinking percentage (% C, measured as the % mass of crosslinker (e.g., bis-acrylamide) in the polymer) of from about 0.1% to about 5%. In embodiments, the acrylamide polymer has a crosslinking percentage with bis-acrylamide of at least 0.5%, at least 0.6%, at least 0.7%, at least 0.8%, or at least 0.9%. In embodiments, the acrylamide polymer has a crosslinking percentage with bis-acrylamide of about 0.5%, about 0.6%, about 0.7%, about 0.8%, about 0.9%, about 1.0%, about 1.1%, about 1.2%, about 1.3%, about 1.4%, or about 1.5%. In embodiments, the acrylamide polymer has a crosslinking percentage with bis-acrylamide of from about 0.5% to about 1.5%, about 0.6% to about 1.4%, about 0.6% to about 1.3%, about 0.6% to about 1.2%, about 0.6% to about 1.1%, about 0.6% to about 1.0%, about 0.6% to about 0.9%, about 0.7% to about 1.3%, about 0.7% to about 1.2%, about 0.7% to about 1.1%, about 0.7% to about 1.0%, about 0.7% to about 0.9%, about 0.8% to about 1.2%, about 0.8% to about 1.1%, about 0.8% to about 1.0%, or about 0.8% to about 0.9%. In embodiments, the acrylamide polymer has a crosslinking percentage with bis-acrylamide of from about 0.8% to about 1.0%. In embodiments, the acrylamide polymer has a crosslinking percentage with bis-acrylamide of about 0.9%.

In embodiments, the gel beads are of a desired or optimal size. In embodiments, the gel beads are of a size such that all of the necessary reactions of a method as provided herein can occur within the gel bead as desired (e.g., enzymes and other reagents can travel inside of the bead and remain active there, and at a desired point, diffuse out). In embodiments, the gel beads are measured as an average diameter of a plurality of the gel beads described herein. In embodiments, the gel beads are at least about 50 microns, at least about 75 microns, at least about 100 microns, at least about 110 microns, or at least about 120 microns in diameter (e.g., average diameter). In embodiments, the gel beads are from about 100 microns to about 200 microns in diameter, about 100 microns to about 175 microns in diameter, about 100 microns to about 150 microns in diameter, about 100 microns to about 140 microns in diameter, about 100 microns to about 130 microns in dimeter, about 100 microns to about 120 microns in diameter, about 110 microns to about 200 microns in diameter, about 110 microns to about 175 microns in diameter, about 110 microns to about 150 microns in diameter, about 110 microns to about 140 microns in diameter, about 110 microns to about 130 microns in dimeter, or about 110 microns to about 120 microns in diameter (e.g., average diameter). In embodiments, the gel beads are about 100, 110, 120, 130, 140, 150, 160, 170, 180, 190, or 120 microns in diameter (e.g., average diameter). In embodiments, the gel beads are from about 100 microns to about 150 microns in diameter (e.g., average diameter). In embodiments, the gel beads are about 120 microns in diameter (e.g., average diameter). In embodiments, the gel beads have a desired degree of uniformity of size (e.g., at least 90% of the gel beads fall within a desired size range, such as any of the ranges provided herein).

In embodiments, the gel beads comprise mRNA capture probes covalently attached to the gel beads. In embodiments, the mRNA capture probes are capable of binding to mRNA released from the cell nucleus within the gel bead such that it does not readily diffuse outside the gel bead. In embodiments, the mRNA capture probes are configured for the capture of mRNA within the gel beads. In embodiments, the mRNA capture probes comprise nucleotides. In embodiments, the mRNA capture probes comprise a nucleotide sequence complementary to a portion of the mRNA within the gel bead. In embodiments, the mRNA capture probes comprise a sequence complementary to the poly-A tail of mRNA within the gel bead. In embodiments, the mRNA capture probes comprise a poly-T sequence (e.g., a sequence of at least 2, 3, 4, 5, 6, 7, 8, 9, 10, or more Ts). In embodiments, the mRNA capture probes act as reverse transcription primers during the reverse transcription step. In embodiments, the mRNA capture probes act as PCR primers.

In embodiments, the method comprises multiple steps of adding DNA barcodes to nucleic acids of the nuclei (e.g., within the gel beads, or, in embodiments, after release from the gel beads). In embodiments, the method comprises at least 3 steps of adding DNA barcodes to the nucleic acids (i.e., adding first DNA barcodes, second DNA barcodes, and third DNA barcodes to the nucleic acids (e.g., cDNA and/or genomic DNA)). The DNA barcodes can be added by any suitable method (e.g., via polymerase chain reaction (PCR), via ligase-based methods (e.g., with T7 ligase), by transposon based methods (e.g., Tn5 transposon), etc.). In embodiments, the method used to add DNA barcodes is selected for optimal properties (e.g., compatibility with later steps, optimal orientation of the DNA barcode, etc.).

In embodiments, the method comprises adding DNA barcodes to nucleic acids contained in a plurality of vessels. In embodiments, it is preferable that each vessel (e.g., a well of a 96-well plate) receives its own unique DNA barcode sequence in order to perform the desired indexing at a later stage of the method. In embodiments, each vessel to which the gel beads are partitioned receives its own unique DNA barcode within an individual DNA barcoding step.

In embodiments, a DNA barcode which is added to a nucleic acid as described herein may comprise nucleic acid sequences which serve other functions (e.g., acting as adapters (e.g., P5 adapters), ligation sites, PCR primer sites, mosaic end sequences, splint handles, etc.). In embodiments, a barcoding sequence of a DNA barcode comprise at least 6, 7, 8, 9, or 10 nucleotides. In embodiments, a barcoding sequence of a DNA barcode comprises at least 10 nucleotides. In embodiments, each barcoding sequence attached to a nucleic acid as provided herein comprises at least 10 nucleotides.

In embodiments, the method comprises partitioning the gel beads to a first plurality of vessels. In embodiments, the gel beads are partitioned such that each of the vessels comprises a roughly equal number of gel beads (and, by extension, gel beads comprising cell nuclei or lysate thereof). In embodiments, the plurality of vessels are wells of a well plate (e.g., a 96- or 384-well plate).

In embodiments, each individual vessel of the first plurality of vessels comprises at least 200 gel beads containing a cell nucleus. In embodiments, each individual vessel of the first plurality of vessels comprises at least 200, 300, 400, 500, 600, 700, 800, 900, or 1000 gel beads containing a cell nucleus. In embodiments, each individual vessel of the first plurality of vessels comprises at least 1000 gel beads containing a cell nucleus.

In embodiments, the method comprises adding a first DNA barcode to the cDNA and genomic DNA. In embodiments, the method comprises adding a first DNA barcode to the cDNA and genomic DNA within the gel beads. In embodiments, adding the first DNA barcode to the cDNA and the genomic DNA comprises transposon barcoding. In embodiments, the transposon barcoding is performed with transposon Tn5. In embodiments, adding the first DNA barcode to the cDNA and the genomic DNA comprises tagmentation. In embodiments, the first DNA barcode comprises a splint oligonucleotide handle (e.g., a sequence of ˜15 nucleotides, optionally positioned to the 5′ end of the barcode portion) and a mosaic end sequence (e.g., a sequence of ˜19 nucleotides position to the 3′ end of the barcode sequence). In embodiments, each of the vessels of the first plurality of vessels has a unique first DNA barcode sequence.

In embodiments, the method comprises pooling and re-partitioning the gel beads to a second plurality of vessels. In embodiments, the gel beads are partitioned such that each of the vessels comprises a roughly equal number of gel beads (and, by extension, gel beads comprising cell nuclei or lysate thereof). In embodiments, the second plurality of vessels are wells of a well plate (e.g., a 96- or 384-well plate).

In embodiments, the method comprises adding a second DNA barcode to the cDNA and genomic DNA. In embodiments, adding a second DNA barcode to the cDNA and genomic DNA within the gel beads. In embodiments, the second DNA barcode is added to the cDNA and the genomic DNA by ligation. In embodiments, the second DNA barcode is added to the cDNA and the genomic DNA by a ligase enzyme. In embodiments, the ligation is performed with a T7 ligase. In embodiments, the second DNA barcode comprises a PCR handle (e.g., a sequence of ˜15 nucleotides positioned to the 5′ end of the barcode portion) and a splint oligonucleotide handle (e.g., a sequence of ˜8 nucleotides positioned to the 3′ end of the barcode portion). In embodiments, each of the vessels of the second plurality of vessels has a unique second DNA barcode sequence.

In embodiments, the method comprises pooling and performing a second re-partitioning of the gel beads to a third plurality of vessels. In embodiments, the gel beads are partitioned such that each of the vessels comprises a roughly equal number of gel beads (and, by extension, gel beads comprising cell nuclei or lysate thereof). In embodiments, the third plurality of vessels are wells of a well plate (e.g., a 96- or 384-well plate). In embodiments, each of the first, second, and third plurality of vessels comprises at least 96 individual vessels.

In embodiments, the method comprises amplifying the cDNA within the gel beads within the third plurality of vessels. In embodiments, the amplifying is performed by PCR. In embodiments, the PCR is performed by at least 2, 3, 4, 5, 6, 7, 8, 9, or 10 cycles.

In embodiments, the method further comprises separating the cDNA from the genomic DNA. In embodiments, separating the cDNA from the genomic DNA comprises forcing the cDNA out of the gel beads. In embodiments, separating the cDNA from the genomic DNA comprises centrifuging the gel beads to form a pellet and removing supernatant containing the cDNA. In embodiments, centrifuging the gel beads forces the cDNA out of the gel beads. In embodiments, the supernatant contains a sufficient amount of the cDNA to allow for subsequent processing, but may not yield all of the cDNA present in the sample. In embodiments, the supernatant contains at least 20%, at least 30%, at least 40%, at least 50%, at least 60%, at least 70%, at least 80%, or at least 90% of the cDNA from the sample. In embodiments, the pellet comprises the gel beads, including the genomic DNA (or a substantial portion of the genomic DNA).

In embodiments, the third DNA barcode is added to the cDNA by polymerase chain reaction (PCR) of the cDNA in the supernatant. In embodiments, the third DNA barcode comprises a P5 adapter (e.g., a sequence of ˜29 nucleotides positioned to the 5′ end of the barcode portion) and a PCR handle (e.g., a sequence of ˜15 nucleotides positioned to the 3′ end of the barcode portion). In embodiments, the third DNA barcode is added to the cDNA by PCR of the genomic DNA.

In embodiments, the method comprises performing bisulfite conversion of the separated genomic DNA. In embodiments, the performing bisulfite conversion of the separated genomic DNA comprises adding bisulfite conversion reagents to the pellet. In embodiments, the method comprises adding a third DNA barcode to the separated genomic DNA. In embodiments, the third DNA barcode is added to the separated genomic DNA after bisulfite conversion. In embodiments, the third DNA barcode comprises a P5 adapter (e.g., a sequence of ˜29 nucleotides positioned to the 5′ end of the barcode portion) and a PCR handle (e.g., a sequence of ˜15 nucleotides positioned to the 3′ end of the barcode portion). In embodiments, the third DNA barcode is added to the genomic DNA by PCR of the genomic DNA.

In embodiments, the method further comprises a gap filling step. In embodiments, the gap filling step is performed to fill gaps formed due to the use of transposon barcoding (e.g., by Tn5). In embodiments, the gap filling step comprises amplifying the nucleic acids in the presence of a 5-methylcytosine dNTP. In embodiments, the gap filling steps preserves barcode integrity during the bisulfite conversion step.

In embodiments, the method comprises sequencing the cDNA and the genomic DNA. In embodiments, the sequencing is performed by next-generation sequencing. Next-generation sequencing platforms include those commercially available from Illumina (RNA-Seq) and Helicos (Digital Gene Expression or “DGE”). Next generation sequencing methods include, but are not limited to those commercialized by: 1) 454/Roche Lifesciences including but not limited to the methods and apparatus described in Margulies et ah, Nature (2005) 437:376-380 (2005); and U.S. Pat. Nos. 7,244,559; 7,335,762; 7,211,390; 7,244,567; 7,264,929; 7,323,305; 2) Helicos Biosciences Corporation (Cambridge, MA) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLID sequencing); 4) Dover Systems (e.g., Polonator G.007 sequencing); 5) Illumina, Inc. as described in U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. Such methods and apparatuses are provided here by way of example and are not intended to be limiting.

In embodiments, the method obtains single cell sequencing data from more cell nuclei than is possible or practical with other methods. In embodiments, the method obtains single cell sequencing data from at least 10,000 cell nuclei. In embodiments, the method obtains single cell sequencing data from at least 10,000, 20,000, 30,000, 40,000, 50,000, 60,000, 70,000, 80,000, 90,000, 100,000, or more cell nuclei. In embodiments, the method obtains single cell sequencing data from at least 100,000 cell nuclei. In embodiments, the single cell sequencing data is both RNA sequencing data (e.g., but sequencing the cDNA) and genomic DNA sequencing data.

### EXAMPLES

### Example 1—Overview of Single Cell DNA Methylation and RNA Sequencing Approach

The disclosure provides a single cell sequencing method that can sequence DNA methylation and RNA from the same cell at the scale of 50,000-100,000 cells using three 96 well plates.

### Process Overview

Provided herein is a new system that can co-sequence DNA methylation and RNA from the same cell at this scale. Existing art with the same DNA methylation and RNA modality can only sequence single cells at a smaller scale (e.g., tens of cells). In embodiments, the technique described herein utilizes a combinatorial indexing concept to increase the cell throughput which has been described in previous art. Additionally, in embodiments, a key innovation is the encapsulation of single cells with lysis buffer and acrylamide monomer in an oil emulsion using a microfluidic device droplet maker. The encapsulated cells are lysed and the acrylamide polymerized into a hydrogel. The encapsulated cells in hydrogel beads then undergo combinatorial indexing and novel library construction chemistries for DNA methylation and RNA sequencing. The approach provided herein describes the first method that involves the encapsulation of single cells or nuclei in hydrogel beads with the associated chemistries. In some instances, similar reactions were previously known in the art, but have been modified to be compatible with a gel bead platform as described herein.

In some instances, a key feature of the platform described is the encapsulation of single cells containing lysis buffer and acrylamide monomer in an oil emulsion using a microfluidic device droplet maker. Reverse transcription primers have 5′ acrydite modifications to co-polymerize with the acrylamide and capture the RNA. After an overnight incubation, each droplet polymerizes into a polyacrylamide bead with the genomic DNA dispersed and intertwined in the polyacrylamide matrix. The acrydite group incorporates the reverse transcription primers to the polyacrylamide back bone. The RNA hybridizes to the reverse transcription primers and are anchored to the gel bead. This polyacrylamide gel bead is accessible to the enzymes critically responsible for cDNA synthesis and combinatorial barcoding. After emulsion breaking, the beads undergo reverse transcription as described in other studies and second strand synthesis overnight (Li et al. 2020).

The DNA and RNA barcoding scheme is in some ways similar to previously published Tn5 based split and pool combinatorial barcoding methods, but has been specially adapted herein for polyacrylamide beads as opposed to nuclei (Domcke, Hill, Daza, Cao, O'Day, Pliner, Aldinger, Pokholok, Zhang, Milbank, Zager, Glass, Steemers, Doherty, Trapnell, et al. 2020; Cao et al. 2019). Briefly, the beads are dispersed into a 96 well plate so that each well contains roughly 200 encapsulated cells or nuclei. Hyperactive Tn5 containing 5′ phosphorylated transposons tagment the beads adding the first DNA barcode using optimized reaction conditions provided herein. The beads are then pooled, washed, and split into a second 96 well plate where the second DNA barcode is ligated to the transposon overhang. Finally, the beads are then pooled, washed, and split into a third 96 well plate. Linear amplification for 10 cycles is used to first amplify the cDNA allowing it to diffuse out of the gel bead to split the cDNA libraries from the gDNA using a PCR primer reverse complement to the reverse transcription primer sequence. The beads are then pelleted and 50% of the supernatant containing the cDNA is exponentially amplified for 7 cycles adding the third barcode to the cDNA. The cDNA reaction is then bead purified using Solid Phase Reversible Immobilization (SPRI) beads at a 0.8×ratio followed by another 10 cycles of PCR using a P5 primer and an i7 primer. Once this reaction is complete, the wells are pooled and 0.8×bead purification is performed twice on the pool.

After linear amplification and extraction of cDNA, the gDNA bisulfite conversion reagent is added to the remaining gDNA for bisulfite conversion. manufacturers protocol for desulphonation was followed with a key modification. At this point, the magnetic beads coat the gel beads which contain the gDNA. Instead of eluting the DNA from the magnetic beads, the magnetic beads along with the gel beads were added to a PCR reaction where the gDNA is linearly amplified for 20 cycles with primers hybridizing to the ligated adapter. This process allows gDNA to diffuse out of the gel bead. The third barcode is added to the gDNA during this linear amplification process. rSAP (shrimp alkaline phosphatase) is then added to the reaction to remove all 5′ phosphates that could potentially interfere with the adaptase protocol. The DNA is then bead purified using SPRI beads at a 1.2×ratio and eluted into the standard adaptase reaction protocol, following the manufacturer's instructions. PCR master mix containing a P5 primer and an i7 primer is then added to the heat inactivated adaptase reaction as described in senmC-seq (Luo et al. 2018). 8 cycles of exponential amplification are then performed. The reaction was then bead purified at a 0.8×ratio followed by another 8 cycles of PCR using P5 and P7 primers. Finally, the wells are pooled and 0.8×bead purification was performed twice on the pool. After purification, the libraries are ready for sequencing.

### Example 2—Design of A Vessel To Immobilize Genomic DNA (gDNA) and RNA

Single cell methods require the compartmentalization of either DNA or RNA during the single cell barcoding steps. In the case of single cell per well methods, the reaction well physically provides this compartmentalization where the nucleic acids of each single cell is given a well specific barcode. For example, in snmCAT-seq the well specific barcode is added to both the DNA and RNA during the post PCR bisulfite conversion (Luo et al. 2022). In the case of combinatorial indexing methods, the cell nucleus provides the compartmentalization during the combinatorial barcoding steps (Mulqueen et al. 2018). Therefore, the success of this technology depends on the single cell compartmentalization of both the DNA and RNA through the combinatorial barcoding steps.

The cell or nucleus must be completely lysed because DNA binding proteins such as nucleosomes only allow the accessible DNA to be barcoded. This blocking of barcoding enzymes by nucleosomes is the basis of existing DNA accessibility combinatorial indexing technologies like sci-ATAC seq. In contrast, whole genome sequencing methods require the inaccessible DNA to also be barcoded. Therefore, these DNA binding proteins must be adequately denatured. For single cell per well methods, single cells or nuclei are fully lysed in the well. In the case of snmCAT-seq, the nuclei are sorted into a reverse transcription buffer that also permeabilizes the nuclei allowing reverse transcriptase to access the nuclear RNA. The thermocycling that accompanies amplification of full-length cDNA and subsequent bisulfite conversion denatures the nucleus and chromatin organization proteins. This process allows for both the DNA and cDNA to be fully accessible to the post bisulfite adapter tagging enzyme, adaptase, theoretically barcoding the full methylome and transcriptome. The challenge for whole genome combinatorial indexing is that the full lysis of DNA binding proteins often results in the lysis of the nucleus. However, the structural integrity of the nucleus is required to compartmentalize the DNA and RNA during combinatorial indexing. In the case of sci-MET, this problem is mitigated by first fixing the cells or nuclei with formaldehyde followed by SDS treatment. This careful balancing of fortifying the nuclear structure yet denaturing some of the DNA binding proteins allows for both increase genome coverage and nucleus integrity. As published, this balanced technique is called nucleosome depletion. Although increased genome coverage is demonstrated, the genome coverage is 5-10 fold lower than single cell per well methods as not all the DNA binding proteins are denatured (Mulqueen et al. 2018).

Herein, the inventors explored different DNA and RNA immobilization and whole methylome accessibility techniques. The inventors first tried to adapt the sci-MET nucleosome depletion technique to reverse transcription, required for transcriptome sequencing. This approach failed because the nucleosome depletion severely compromises the nuclear integrity causing 90% of the nuclei to be destroyed after reverse transcription. The need for a more robust vessel with much higher structural integrity compared to the nucleosome depleted nuclei was therefore identified. This led to the development of a simultaneous cell encapsulation and lysis within hydrogel beads method. With inspiration from previously published combinatorial indexing RNA sequencing methods, in-nuclei reverse transcription was performed followed by nuclei encapsulation and lysis by high concentrations of SDS and proteinase K (Rosenberg et al., n.d.; Plongthongkum et al. 2021; C. Zhu et al. 2019). The microfluidic hydrogel encapsulation approach described herein offers the added advantage of using strong protein denaturation buffers to ensure the complete denaturation of DNA binding proteins, and the robust compartmentalization of nucleic acids. This high stability allows for the easy incorporation of reverse transcription and additional barcoding enzymes to allow for the development of a 3-level WGBS and RNA co-sequencing platform.

Through DNA staining and imaging, the adequate lysis of DNA binding proteins could be confirmed. However, the immobilization of RNA required the screening of different hydrogel structures. Simply, the RNA is over 50,000× shorter in length than DNA which allows the RNA to easily diffuse out of the hydrogels. Thus, three hydrogel structures were assessed: agarose gel beads, polyethylene glycol (PEG) gel beads, and finally polyacrylamide gel beads. The polyacrylamide gel beads offered the best solution as reverse transcription primers could be modified with an acrydite group. During gel polymerization, this acrydite modified primer covalently anchors the cDNA to the polyacrylamide matrix. The long DNA is intertwined in the polyacrylamide gel matrix. Thus, this structure successfully immobilizes both the fully accessible DNA and RNA which enables whole genome and transcriptome combinatorial indexing. The success of this approach was demonstrated by performing single cell whole genome and transcriptome sequencing on a mixture of human and mouse cells. After sequencing, cell barcodes that contained only human or mouse reads were observed.

### Methods and Results

### Nucleosome Depletion Adaptation

Initial approaches were inspired by two combinatorial indexing techniques: sci-RNA seq and sci-MET seq. The goal was to combine the in-nuclei reverse transcription technique to process the RNA and the nucleosome depletion technique for whole genome barcoding. Nucleosome depletion followed by reverse transcription to generate a nuclear structure containing nucleosome depleted DNA and cDNA was first attempted. Using a two-level indexing scheme, 1000-2000 nuclei would first be FACS sorted into a 96 well plate where Tn5 would be used to add the first cell barcode. The nuclei would then be pooled and then 10-20 nuclei per well were FACS sorted into as second 96 well plate where PCR indexed adapters reverse complement to the Tn5 adapter sequences would be used to add the second bell barcode, completing the combinatorial indexing process.

The primary issue with nucleosome depletion was the integrity of the nuclei following depletion. This was assessed by first staining the nuclei with a standard DNA stain, DAPI. Intact nuclei contain higher levels of DAPI compared to nuclear/chromosome debris. The number of intact nuclei and nuclear debris can be measured using FACS and plotting the DAPI fluorescent intensity. Briefly, the FACS machine measures the forward and side light scattering and DAPI fluorescent intensity of the nuclei or debris. A gate is manually drawn to distinguish nuclei from debris. Particles with sufficient DAPI fluorescence are collected as nuclei whereas all other particles of lower fluorescence are assumed to be debris. For clarity, the DAPI gate is labeled in each plot. Freshly isolate nuclei are first sorted to identify a baseline DAPI fluorescent intensity. Examining the DAPI signal plot, most particles have high DAPI signal and a threshold of 1000 460/50 [405] is used to differentiate intact nuclei and debris. Next, nucleosome depleted nuclei are sorted using the same DAPI fluorescent threshold. Clearly, the nucleosome depletion process generates large amounts of nuclear debris as a large population of particles have low DAPI fluorescence.

This study encapsulates the immense difficulty in recovering intact nuclei from each required reaction. After nucleosome depletion, the SDS and formaldehyde are removed by pelting the nuclei and removing the supernatant. The nuclei were then resuspended in reverse transcription buffer and incubated at 55 C for 10 minutes, following the sci-RNA seq reverse transcription protocol. Afterwards, the nuclei are pelleted and resuspended in PBS containing DAPI for FACS. In these experiments, only 1% of nuclei after nucleosome depletion and reverse transcription from FACS. Thus, this nucleosome depletion approach was deemed unfeasible.

In-Nuclei cDNA Generation and Agarose Gel Encapsulation

The immense difficulty in handling nucleosome depleted nuclei motivated a new approach inspired by SiC-seq where single microbes were encapsulated in agarose micro-sized gel beads, lysed with SDS and proteinase K, and finally individually barcoded using a system of microfluidic devices (Lan et al. 2017). The agarose gel bead encapsulation and lysis approach to immobilize the DNA and cDNA of nuclei after in-nuclei reverse transcription was thus attempted to be adapted. The microfluidic device used to achieve this encapsulation was custom designed by PhD student Andrew Richards, and is described in his dissertation from the University of California San Diego which can be found at scholarship.org/uc/item/4zk292pm, the contents of which are herein incorporated by reference. The specific microfluidic device engineering and encapsulation protocol is detailed in the supplemental methods. To summarize some key features, the device is configured to create gel beads encapsulating cell nuclei or lysate thereof at a size which optimizes efficient diffusion of DNA barcoding reagents (e.g., Tn5, ligase, etc.) through the gel bead. This is accomplished by providing gel beads preferably having a diameter of about 100 to about 150 microns. In order to create beads of this size, the device has a depth of about 30 microns and a junction width of about 50 microns. Such smaller bead sizes allow for better sensitivity (e.g., in terms of sequenceable DNA molecules or information content per cell). One potential drawback of such an approach is the requirement of a high concentration of cells or nuclei necessary to achieve a sufficiently high encapsulation rate for subsequent combinatorial indexing. Thus concentrations of, for example, at least 1000 cells or nuclei per microliter (typically about 3000 cells or nuclei per microliter) are preferred. Use of such higher concentration (relative to other techniques, such as BAG-Seq (as described by Li, Siran et al., Genome Res. 2020. 30:49-61; doi: 10.1101/gr.253047.119) can cause higher concentrations of particulates and thus increase the chances of device failure (e.g., due to clogs of the microchannels). However, this risk can be overcome by suitable configuration of the device used to make the gel bead encapsulations, such as by applying a coating to the device (e.g., a water-resistant coating such as Aquapel®).

With inspiration from InDrops and Drop-seq, the microfluidic device encapsulates single cell or nuclei within oil droplets. In the instant adaptation, a suspension of single nuclei in low melting temperature agarose kept at 37 C is created. This mixture is input through the encapsulation device along with 0.5% SDS and 0.016 U/μL proteinase K. A space heater is used to warm the encapsulation device and fluid reservoirs to 37° C. to prevent gelling of the agarose prior to encapsulation.

Agarose demonstrates robust structural integrity when exposed to high concentrations of SDS and proteinase K. The size of a typical nucleus is roughly 1-5 microns while the gel bead is roughly 120 microns in diameter. The DNA content of gel beads can be visualized by staining them with DAPI. The robust denaturation of DNA binding proteins can also be confirmed by observing the diffusion of DNA throughout the hydrogel matrix.

The encapsulation of single cells or nuclei can be described by a Poisson probability distribution as described in previous cell encapsulation methods such as InDrops and Drop-Seq (Klein et al. 2015; Macosko et al. 2015). Using the volume of the gel bead and a goal of roughly 10% of beads occupied by single nuclei, 90% of beads empty, and negligible numbers of beads containing multiple nuclei, Poisson distribution was used to predict the required concentration of nuclei prior to encapsulation as 3000 nuclei/μL. After encapsulation, the occupancy of the beads is visually calculated by counting the number of empty beads and stained beads. With 10% of the beads DAPI positive, it was verified that the encapsulation method follows a Poisson distribution as described previously (Klein et al. 2015; Macosko et al. 2015).

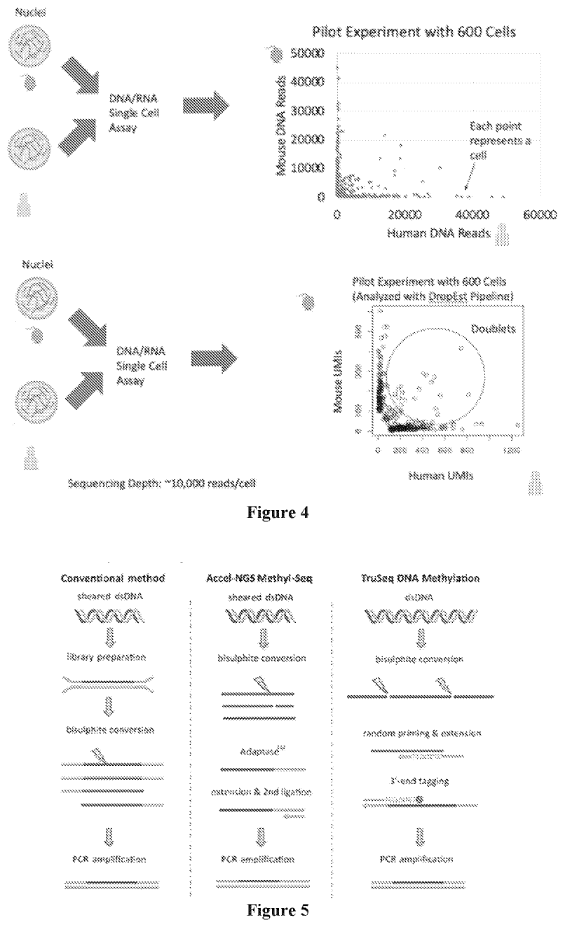

In the instant adaptation, nuclei are first freshly isolated from cultured cells and then undergo the reverse transcription and second strand synthesis reactions previously described in sci-RNA seq. Afterwards, the nuclei are washed once with nuclei isolation buffer without NP-40 and filtered through a 30-micron filter to remove nuclei aggregates. The nuclei were then resuspended in a low melting temperature 1.5% agarose PBS mixture pre-warmed to 37° C. to prevent gelling. Encapsulation was then performed using a microfluidic device described previously. To keep the agarose from polymerizing, the encapsulation was performed with a space heater to keep the agarose on the device and in the fluid reservoirs at roughly 37 C. FIG. 4 illustrates the general steps prior to gel bead formation. Post encapsulation, the agarose gel beads were removed from the emulsion using previously described methods (Klein et al. 2015). Briefly, the emulsion oil was carefully removed, and the emulsion was broken using 20% 1H, 1H,2H,2H-Perfluorooctan-1-ol (PFO) v/v in HFE7500. The beads were then washed with 1% Span80 in hexane followed by 0.1% tween 20 in Tris HCl ph=7.5. The agarose beads were then wash twice in H₂O and then stained with DAPI to calculate occupancy and establish input amounts for DNA and cDNA library generation.

Due to its length the DNA could safely be assumed to be immobilized in the gel matrix. However, the cDNA could freely diffuse out of the gel bead. To assess this possibility, Tn5 was first used to tagment the cDNA and then amplified the cDNA using PCR primers reverse complement to the reverse transcription primer and the Tn5 adapter. qPCR was then used to quantify the amount of encapsulated cDNA compared to a positive control: cDNA in a tube and a negative control: no cDNA. From this experiment, it was observed that cDNA was not retained inside of the gel bead as the amplification dynamics of the agarose gel bead samples matched that of the negative control. Although the agarose gel bead structure was relatively simple to work with due to the ease of nucleic acid extraction under heat, the large pore sizes (estimated to be between 100-200 nanometers) resulted in loss of the cDNA, thus indicating further optimization was needed.

### PEG Acrylated Gel Formation

The next approach attempted was to lower the pore size of the hydrogel. A polyethylene glycol (PEG) hydrogel was considered inspired by a virtual microfluidic method by the Paul Blainey group (Xu et al. 2016). The pore size diameter of this hydrogel was reported to be 25 nanometers, drastically lower than the pore size of the agarose gel. Using a flexible chain molecular model, it was estimated that DNA above 60 bases could potentially be immobilized by this gel (Pluen et al. 1999). With a protocol like the agarose one described previously, it was planned to encapsulate nuclei with this polymer. Resuspending the nuclei in 8-arm PEG and co-encapsulated the nuclei with a thiol-PEG crosslinker dissolved in 0.5% SDS was attempted. Proteinase K was removed since proteinase K destroys the ester bonds formed during gel polymerization. To test the retention of DNA within these gel beads, it was first tried to encapsulate a DNA ladder in the size range of cDNA and washed these gel beads. It was then dissolved the gel beads with proteinase K and then ran a polyacrylamide gel electrophoresis experiment with the unencapsulated ladder. After DNA staining and imaging the gel, the ratio of fluorescent intensity of the DNA unencapsulated ladder and encapsulated ladder was used to estimate the loss of encapsulated DNA based on size. Unfortunately, clear loss of DNA was noticed within the typical size range of cDNA (800-2000 bases in length), suggesting this approach was also sub-optimal.

From these experiments, it was theorized that the amount of bead pelleting and washing post encapsulation to break the emulsion adds a fluidic and mechanical force that will cause the cDNA to diffuse out of the gel bead. Thus, it was concluded that cDNA can only be immobilized chemically, ideally with a covalent bond

### Polyacrylamide Gel Formation

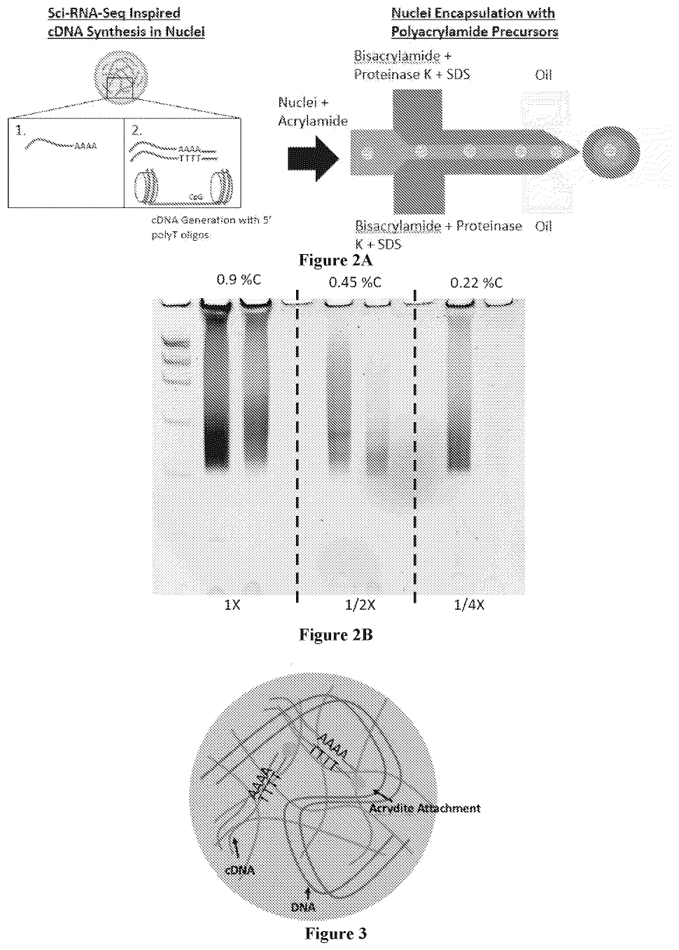

The next approach took inspirations from a version of polony sequencing developed by the George Church group (Mitra and Church 1999). In this iteration, a polyacrylamide hydrogel approach was designed where the reverse transcription primer contains an acrydite modification allowing it to be covalently anchored to the gel bead matrix during gel bead polymerization. Like previous approaches, cDNA is first synthesized in-nuclei using an adapted version of the sciRNA-seq protocol with acrydite modified reverse transcription primers. The nuclei are then pelleted and resuspended in an encapsulation buffer containing acrylamide monomers. The nuclei are then encapsulated with protease K, SDS, and a bisacrylamide crosslinker as illustrated in FIG. 2A. This solution proved to be the correct approach to immobilize both the DNA and cDNA. A crosslinker percentage (% C, calculated as the mass percentage of bis-acrylamide crosslinker in the polyacrylamide gel bead) of 0.9% C was observed to produce optimal results. A shown in FIG. 2B, a crosslinker percentage of 0.45% C (depicted as “½×”) yielded only adapted dimers and 0.22% C (“¼×”) yielded no DNA library at the end of the process. Conversely, the 0.9% C yielded a uniform library of polynucleotides of different lengths ready for sequencing, indicating a robust library preparation. The polyacrylamide hydrogel is also structurally resistant to SDS and proteinase K. Through the acrydite modification, the synthesized cDNA using the reverse transcription primer is covalently anchored to the polyacrylamide matrix (FIG. 3).

To assess the efficiency of acrydite incorporation, a polyacrylamide electrophoresis experiment was performed where the polyacrylamide gel beads were directly added to the wells of the gel during electrophoresis. In parallel, a denaturing polyacrylamide electrophoresis experiment was performed where the cDNA within the polyacrylamide beads was first denatured in urea at 98° C. for 5 minutes and then placed on ice for 2 minutes. These gel beads were then directly added to the wells of a polyacrylamide gel infused with urea to keep the cDNA denatured. Because only one strand of the cDNA is anchored to the gel bead, the complement strand will migrate through the polyacrylamide gel infused with urea after urea denaturation of the cDNA. In contrast, the undenatured cDNA will not migrate through the gel during electrophoresis. In this experiment, analysis of the resulting PAGE gels did not identify any cDNA eluting from the undenatured bead, whereas the cDNA was observed in the denatured bead, indicating robust covalent anchoring of cDNA within the gel bead.

sciGel Version 1: gDNA and RNA Sequencing Library Formation Protocol

Having identified a bead structure capable of retaining cDNA, a combinatorial indexing scheme was tested using this gel bead structure by processing an even mixture of mouse and human nuclei. In this way, the success of combinatorial indexing could be revealed after sequencing the library. Nuclei barcode combinations that contain only human reads or mouse reads suggest single cell resolution. Barcodes that contain both human and mouse reads are considered mixed. In an even mouse and human mixture, the barcode collision rate is estimated as two times the mixed rate as mouse-mouse and human-human doublets cannot be measured. The detailed nuclei isolation protocol can be found in the supplementary methods section. Briefly, cDNA is synthesized in-nuclei like in previous designs. The nuclei are then simultaneously encapsulated and lysed using the same microfluidic device. After an overnight polymerization, the emulsion is broken to extract the gel beads. The beads are then stained with DAPI and the occupancy and concentration of nuclei are calculated. 100-200 nuclei/well are added to a 96 well plate and then tagmented with Tn5 mixture loaded with two different transposon sequences now referred to as Tn5 A and Tn5 B. This Tn5 A is well specific and contains the first nuclei barcode to the DNA and cDNA while Tn5 B is simply a PCR handle. The beads are then pooled and washed twice with 0.1% tween20 in Tris-HCl pH=8 followed by two washes in H₂O by pelleting the beads 300×g for 2 minutes. The beads are then counted with a hemocytometer and then 10-20 nuclei are split into a second 96 well plate. The Tn5 was denatured with 0.1% SDS and then quenched with 2% Triton-X. PCR master mix was then added to each well with a PCR primer reverse complement to the cDNA capture primer. Because polyacrylamide is extremely stable, it was discovered that in-gel PCR had to be used to extract both the gDNA and cDNA.

The cDNA was then linearly amplified for 10 cycles. Then, a well specific PCR primer reverse complement to Tn5 A and a PCR primer reverse complement to Tn5 B was added. Both the cDNA and gDNA was then exponentially amplified together for 6 cycles. Each reaction was then individually bead purified with SPRI beads at a 0.8×ratio. The eluted, DNA/cDNA was then evenly split into two separate plates. One plate finishes the amplification of cDNA by adding a P7 primer reverse complement to the reverse transcriptase primer and a P5 primer reverse complement to the Illumina P5 sequence. The other plate finished the amplification of DNA by adding PCR primers reverse complement to the Illumina P5 and P7 sequences. After amplification is complete, both the DNA and cDNA libraries are separately pooled and bead purified twice with SPRI beads at a 0.8×ratio. PAGE was then performed to confirm successful library generation illustrated by a smear between 200-600 bp. The libraries were sequenced with a MiSeq.

### Accompanying Bioinformatic Methods

Briefly, libraries were first demultiplexed using index 1 used to distinguish cDNA libraries from DNA ones using bcl2fastq. Deindexer was used to demultiplex both DNA and cDNA libraries into individual cell barcode files based on the Tn5 and PCR barcodes. The files were then concatenated while retaining the cell barcode in the read ID of the fastq file. Adapter sequences were then trimmed from both the DNA and cDNA concatenated files using cutadapt. The DNA library was aligned to a concatenated human and mouse genome using bowtie2.

Similarly, the RNA library was aligned to a concatenated human and mouse genome using STAR. The dropEst package was then used to collapse the cDNA UMI space and generate a cell barcode x gene counts matrix. The amount of human and mouse reads for each cell barcode was then quantified and plotted.

### Species Mixing Results

FIG. 4 illustrates the workflow described previously with the species mixing plot shown. Here, each point is a recovered cell or nuclei barcode and the coordinates of each point quantify the amount of human and mouse reads for that specific barcode. It was observed that points that aligned with both the human and mouse axes indicating the presence of single cells for both the DNA and cDNA libraries. However, about 25% of the barcodes were mixed resulting in a high barcode collision rate of about 50%. This means that about half of the datasets were single cells while half of the datasets were doublets. Despite this high collision rate, a promising result that the polyacrylamide gel encapsulation scheme with acrydite modified reverse transcription primers could result in single cell gDNA and RNA libraries co-sequenced from the same cell was demonstrated.

### Conclusion

The development of an RNA and DNA co-sequencing platform using polyacrylamide gel beads as the combinatorial indexing container was described. Acrydite modified reverse transcription primers were used as the cDNA immobilizing scheme while DNA was immobilized by the polyacrylamide mesh. This final design was arrived at by screening a variety of nucleic acid containers. The most straightforward approach was to leverage the nucleosome depleted nuclei, but this approach was unreliable due to the low structural integrity of these nuclei. To increase the structural integrity of the nucleic acid container, a hydrogel encapsulation approach was attempted. Agarose was first used but it was observed that cDNA easily diffused out of the gel bead.

The pore size was then lowered using a PEG acrylate gel, but still it was observed that a noticeable amount of nucleic acid products in the size range of cDNA was lost. This led to the insight of using covalent anchoring of the cDNA using acrydite modified reverse transcription primers and a polyacrylamide gel bead vessel. The potential of this platform was then demonstrated by designing a combinatorial indexing scheme adapted from previous work to co-sequencing DNA and RNA libraries. While the barcode collision rates from this experiment were somewhat high likely due to the inherent inaccuracies of estimating the input of 10-20 nuclei into the second 96 well plate during combinatorial indexing, it is predicted that such an approach could be readily optimized. Unlike previously combinatorial indexing methods, the gel beads are too large to be sorted using readily available methods, and so some wells in the second indexing plate may contain multitudes higher or lower numbers of nuclei causing higher than expected barcode collisions. Future optimization could potentially include using a fluorescence activated cell sorting (FACS) machine with custom settings to account for the additional size of the gel beads, the innovation of a third level of combinatorial indexing, or other potential optimizations.

This powerful platform has the potential to assess copy number variations and RNA from the same cell or nuclei. This may be particularly relevant in the study of high-risk neuroblastomas where copy number increase of the MYCN oncogene on chromosome 2p occurs in 20% of them (Dzieran et al. 2018). This MYCN copy number variation typically results in poor prognosis (Dzieran et al. 2018). The single cell gDNA sequencing of neuroblastoma tumors could bioinformatically isolate MYCN copy number amplified tumor cells and profile. The whole transcriptomes of these MYCN amplified tumor cells could then be profiled to potentially identify therapeutic pathways to specifically target MYCN amplified tumor cells.

### Example 3—Single Cell Methylation and RNA Integration

Described herein is the development of a novel single cell methylation library construction protocol that was designed specifically to tackle the challenges of performing bisulfite conversion in polyacrylamide gel beads. The gold standard method to perform single cell methylation sequencing employs harsh bisulfite conversion chemistries. There are a few main challenges in developing the protocol around these chemistries. Firstly, bisulfite conversion converts unmethylated cytosine to thymine which results in the cytosines in the unique molecule identifier (UMI) incorporated in the reverse transcription capture primer, required for single cell RNA sequencing, to also be converted to thymine. The cytosines in the Tn5 adapter sequences are also converted resulting in a lowering of the PCR primer annealing temperatures which causes extensive off-target PCR products. Secondly, bisulfite conversion produces extensive DNA fragmentation (Ahn et al. 2021). For the cDNA library, fragmentations result in the complete loss of the molecule because one end contains the cell barcode while the other end contains the UMI. Because Tn5 inserts in two ends of the DNA library, fragmentations result in the loss of one of the adapters which prevents the addition of Illumina sequencing adapters during PCR. Thirdly, most of the DNA is still contained inside the polyacrylamide beads during the bisulfite conversion process. Typically, DNA is eluted from either a silica column or magnetic bead once bisulfite conversion is completed. Because the DNA has not been extracted yet, a method that ensure that the gel beads are also moved to the steps beyond the bisulfite reaction is needed.

The methods to protect the Tn5 PCR adapter sequence and the RNA UMI were first addressed. To complete the Tn5 reaction, the Tn5 must be denatured with 0.1% SDS (Picelli, Björklund, et al. 2014). After denaturation, the DNA is fragmented into double stranded products with a 5′ overhang. This complementary sequence of the overhang can be synthesized with a high fidelity polymerase that is resistant to SDS by extending the recessed 3′ end using the 5′ overhang as the template strand. This process is called gap filling. To protect the Tn5 adapter sequence from the cytosine to thymine conversion, a custom dNTP mixture was created where the cytosine is replaced with methylated cytosine. Thus, the newly synthesized DNA from the recessed 3′ end through the Tn5 adapter contains methylated cytosine. These methylated cytosines are not converted during bisulfite conversion, retaining the original Tn5 adapter sequence for PCR. To protect the cDNA UMI, the cDNA was linearly amplified using a single PCR primer that hybridizes to the reverse transcription capture primer using the same PCR reaction mix to perform gap filling. This process incorporates methylated cytosine to the newly synthesized cDNA products which protects the whole cDNA strand including the UMI from the cytosine to thymine conversion.

To address the DNA fragmentation issue, optimization of the cDNA linear amplification prior to bisulfite conversion was attenoted so that the subset of cDNA that remains could potentially still reflect the original cDNA library complexity. The gDNA library cannot be similarly amplified prior to bisulfite conversion as the original methylated cytosine profile would be altered. Thus, different post bisulfite adapter tagging methods like scnmC-seq were explored to add an adapter sequence to the 3′ end of all the DNA sequences post bisulfite conversion to enable the final PCR required to add Illumina sequencing adapters (Callaway et al. 2021). However, crucial modifications to allow extraction of the DNA form the gel beads prior to the single-end ligation reaction were made. In the previous example, the method of DNA extraction from the polyacrylamide beads requires a combination of PCR and passive diffusion of DNA products from the gel bead. Thus, a linear amplification PCR step after bisulfite conversion was designed using the protected Tn5 adapter sequence as the priming sequence and uracil tolerant polymerase to extract the DNA from the gel bead. This PCR is distinctive to this method because the template is gel beads coated in the magnetic beads used in the Zymo EZ-96 DNA Methylation MagPrep kit. This modification is required for high DNA library complexity as most DNA sequences are still trapped inside the gel bead.

To test the success of this method, lambda phage DNA was spiked in to ensure that the bisulfite conversion efficiency was 99%. The library was then sequenced to shallow depths to assess the mapping rate to in-silico bisulfite converted genomes. After identifying the best mapping software and settings, the methylation data around reference methylation features were binned to validate the methylation dynamics expected around those features.

### Methods and Results

Adapting Post Bisulfite Conversion PCR Adapter Addition Techniques to sci-Gel

The cytosine to thymine conversion and fragmentation of DNA during bisulfite conversion poses significant library design challenges. FIG. 5 illustrates several common WGBS library construction methods. To circumvent the cytosine to thymine conversion of library adapter sequences, conventional bisulfite sequencing involves the addition of methylated adapters. Methylated adapters are typically much more expensive than unmethylated ones. In addition, fragmented sequences resulting from the bisulfite conversion are unrecoverable. The highest library complexity bisulfite sequencing methods involve the addition of adapters post bisulfite conversion which typically involves random priming. At the single cell level, the most effective method was demonstrated in scnmC-seq which first involves cell lysis and bisulfite conversion. Then, an initial random priming and extension step like the TruSeq method is performed to synthesize a complementary strand of DNA using the uracil resistant and strand siplacing polymerase, klenow exo-. The strand synthesized by the random primer is then tagged on the 3′ end with an adapter using the adaptase protocol. Illumina sequencing primers are then added to this product using PCR primers complementary to the random primer PCR handle and adaptase adapter (Luo et al. 2018).

sci-MET takes a slightly different approach. After bisulfite conversion, a random priming and extension step like scnmC-seq is also used. However, this random priming is performed three additional times to increase library complexity. The Illumina sequencing adapters PCR uses primers reverse complementary to the Tn5 adapter and the random priming sequence PCR adapter. The Tn5 adapter sequence is designed to be cytosine depleted and is therefore unchanged through the bisulfite conversion.

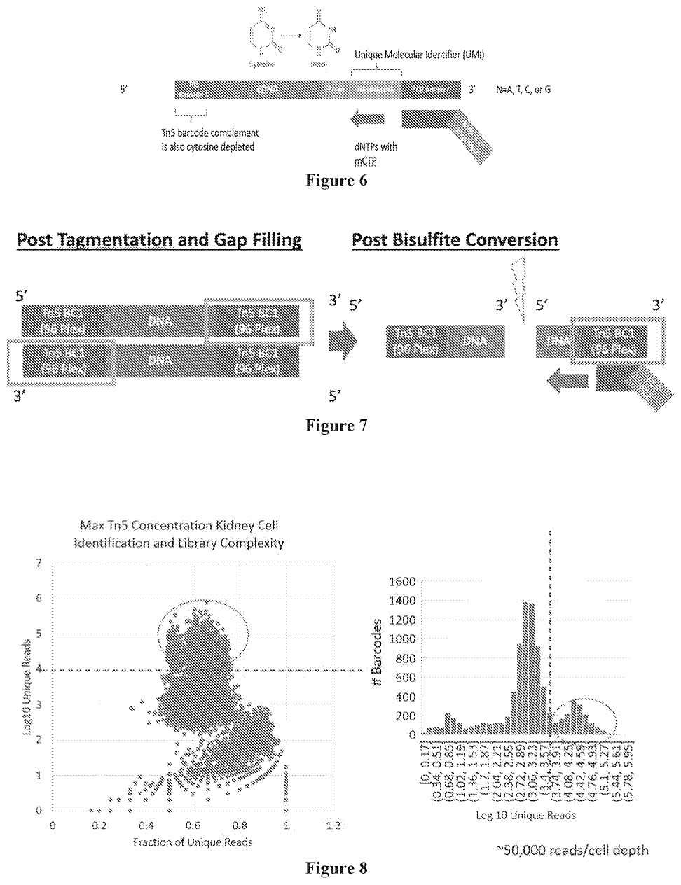

The instant methods use a different approach. FIG. 6 illustrates the cDNA library structure prior to bisulfite conversion. Transcriptome sequencing requires the use of UMIs that can clearly distinguish between PCR duplicates and natural gene expression. The design of the UMI is a random sequence of all bases. However, the bisulfite conversion would mutate the UMI by converting the unmethylated cytosine to thymine. Therefore, it was necessary to linearly amplify the cDNA with methylated cytosines prior to bisulfite conversion to protect the UMI sequence using a PCR primer that is reverse complement to the reverse transcription primer with a cytosine depleted handle. Post bisulfite conversion, it was also necessary to design a non-random priming technique since random priming of the cDNA would likely not contain the UMI sequence.

The second problem with a random priming protocol is that the gel beads are still intact post bisulfite conversion. As discussed previously, the DNA needs to be sufficiently amplified to extract the DNA from the gel beads. A post bisulfite linear amplification scheme was designed where the transposon sequence is first gap filled with methylated cytosines instead of unmethylated cytosines. Instead of eluting the DNA from the magnetic beads per the manufacturer's protocol, the magnetic beads containing intact gel beads are transferred to the linear amplification reaction with PCR primers reverse complement to the gap filled transposon sequence that was protected from bisulfite conversion. FIG. 7 illustrates this linear amplification process. In the most optimized versions of this protocol, the DNA is linearly amplified for 20 cycles with barcoded primers containing the second cell barcode to complete the combinatorial indexing process and sufficiently extract the DNA from the gel beads. The library is then split where the cDNA is exponentially amplified with PCR primers reverse complement to the cytosine depleted PCR adapter on the reverse transcription primer side of the library and the transposon sequence.

Unfortunately, more cDNA was lost during the bisulfite conversion process than expected. It was originally envisioned that the linear amplification of cDNA prior to bisulfite conversion could compensate for the loss of cDNA due to fragmentation. However, approximately 99% of the cDNA was lost. As a result, exponentially amplifying the cDNA prior to bisulfite conversion or splitting the cDNA and gDNA libraries prior to bisulfite conversion as potential solution was explored (see later Examples).

For the DNA library, two different post bisulfite adapter tagging methods were tried to attempt to save costs since the adaptase reaction has an expensive cost of about $20 per reaction. Inspired by a single end ligation design utilizing a modified oligo with 5rapp, the ligation efficiency between this design and the adaptase reaction was assessed (Wu and Lambowitz 2017). However, the adaptase kit (Swift Biosciences™) demonstrated substantially higher ligation efficiency.

After the ligation of the DNA libraries with the adaptase adapter, final PCR to add Illumina sequencing adapters was performed.

sciGel Version 2: Single Cell Methylome Library Formation Protocol

To validate the success of the WGBS method and assess the performance of the assay, single cell WGBS was performed on a colorectal cancer cell line HCT116 and a human kidney tissue. In summary, nuclei undergo reverse transcription, were then encapsulated, 100-200 were split into a 96 well plate and barcoded with Tn5, then 10-20 encapsulated nuclei were split into a second 96 well plate following the same barcoding scheme as described in the whole genome and whole transcriptome co-sequencing assay described in the previous example. The adaptation for methylome sequencing has a few adaptations. PCR reaction mixture was modified substituting cytosine for methylated cytosine. A cytosine depleted cDNA primer reverse complement to the reverse transcription primer is added. Gap filling takes place as previously followed by 10 cycles of cDNA linear amplification. Bisulfite conversion reagent is then added to each well according to the manufacturer's protocol. The samples are then incubated at 98° C. for 8 minutes and 65° C. for 3.5 hours and then kept at 4 C overnight following the standard bisulfite conversion protocol by the manufacturer. Magnetic beads and binding buffer were then added to the bisulfite conversion mixture and transferred to a deep well 96 well plate. The manufacturer's protocol was then followed through the desulphonation step with a modification.

After drying the magnetic beads for 25 minutes, 20 μL was added to each well and then incubate at 55° C. for 5 minutes. Instead of placing the deep well plate back on the magnetic rack, the sample including the magnetic beads were transferred to a 96 well plate. KAPA HiFi Uracil PCR master mix was then added each reaction along with a well specific PCR barcoded primer that is reverse complement to the Tn5 adapter. Linear amplification of the DNA and cDNA then occurs in the presence of the magnetic beads for 20 cycles. Afterwards, rSAP was used to remove the phosphates from residual mosaic end sequences. The samples were then bead purified with SPRI beads at a 1.2×ratio and eluted into a new 96 well plate. Half of the volume was transferred to a new 96 well plate where KAPA HiFi was used to finish amplifying the cDNA library with PCR primers reverse complement to the cytosine depleted cDNA adapter on the reverse transcription side of the library and Illumina P5 sequences. The DNA half of the library was then incubated at 98 C for 3 minutes quickly followed by incubation on ice for 2 minutes to ensure single stranding of the library. The manufacturer's protocol for the adaptase reaction was then performed. After heat inactivation of the adaptase enzymes, KAPA HiFi was used to finish amplifying the DNA library with PCR primers reverse complementary to the adaptase adapter and the Illumina P5 sequences. All the 96 wells for the DNA and RNA plates were pooled and then bead purified with SPRI beads at a 0.8×ratio twice to prepare the library for sequencing. PAGE was performed to check the quality of the library with an expected smear between 200-600 bases. The libraries were sequenced with a MiSeq. The detailed version of this protocol and sequencing scheme is in the supplementary methods. Although the cDNA library was created, it was decided not to sequence it due to the low library complexity due to the loss of cDNA from bisulfite conversion as discussed previously.

The below table shows that strong alignment rates were achieved for the bisulfite converted libraries and 99% bisulfite conversion efficiency which is comparable to existing methods. To assess the biological relevance of the technology, the HCT116 methylome data was pooled and binned across the genomic coordinates of HCT116 H3K4Me3 histone marks based on reference ChIP-seq data. This histone mark is typically hypomethylated and is nearby highly expressed genes (Sharifi-Zarchi et al. 2017). The expected hypomethylation dynamics associated with this feature were observed (data not shown). This validated the integrity of the novel WGBS protocol described herein.

Alignment rates assessed by different library preparation conditions and alignment software. The bisulfite conversion efficiency based on a lambda phage DNA spike-in construct

| | | | Alignment Rate/Bisulfite |
|---|---|---|---|
| Tn5 Concentration | | Alignment Method | Conversion Efficiency |
| 0.1 | mg/mL | BW | 0.79, 99% |
| 0.05 | mg/mL | BWA | 0.68, 99% |
| 0.1 | mg/mL | Bismark PBAT | 0.7, 99% |
| 0.05 | mg/mL | Bismakr PBAT | 0.72, 99% |

Next, the library performance at the single cell level for both the HCT116 and kidney tissue libraries was assessed. The table below summarizes the alignment rates for both libraries showing high alignment rates using the Bismark bowtie2 based method. The CH Methylation level was also low which is expected for terminally differentiated tissue.

| | Alignment | % CH |
|---|---|---|
| Library | Rate | Methylation |
| Single Cell Methylome Kidney Library | 63% | 1.70% |
| (averaged over 2 libraries) | | |
| Single Cell Methylome Cell-Line Library | 62% | 1.50% |
| (averaged over 2 libraries) | | |
| Unmethylated DNA Spike-In | — | 1% |

The number of unique reads against the fraction of unique reads to identify the cells from empty barcodes (FIG. 8). Roughly 90% of the barcodes are empty in combinatorial indexing schemes. Thus, barcodes containing single cells can be discriminated by visually identifying a subset of barcodes with high library complexity (Mulqueen et al. 2018; 2021).

Cells with over 100,000 reads were selected for further downstream analysis. Using pre-seq, the maximal library complexity of the library to 1-2M reads per cell was estimated. The table below summarizes how the WGBS library described herein compares to existing methods. Currently, the instant method exhibits similar performance compared to existing single cell high throughput methods. With further optimizations, it is believed possible to vastly improve the library complexity to approach the library complexity of single cell per well methods.

| | Mapping | Duplication | Sequence | Number of Unique |
|---|---|---|---|---|
| Method | Rate | Rate | Depth/Cell | Reads/Cell |
| PBAT (Hui et al) | 37% | 26% | 1-2M | — |
| scM&T | 18% | 18% | 11.5M | 3M |
| (Angermueller et al) | | | | |
| Sc-nmC (Luo et al) | 50% | 48% | 5.5M | 1.3M |
| sc-BS | 11% (Usable | 11% (Usable | 6M | 673K |
| (Farlik et al) | Reads) | Reads) | | |
| Sci-MET | 67% | 66% | ~200K | ~1-2M (estimated |
| (Mulqueen et al) | | | | w/Pre-seq) |
| Sci-Gel-M (Single | ~60% | 30% | ~100K | ~1-2M (estimated |
| Cell Library) | | | | w/Pre-seq) |

Using the cells with over 100,000 reads/cell, the reads were binned into 1 megabase windows. This large window size reflects the sparsity of the WGBS datasets. Roughly 200 CH positions in each bin per cell were observed. Surprisingly, the number of CG positions is roughly 10 fold less. Since CH methylation is very low in kidney tissue, it was concluded that this coverage was too sparse to confidently perform additional analysis on this dataset. Attempting to perform single cell clustering using kidney tissue is likely impractical without RNA information.

### Accompanying Bioinformatic Methods

These additional analyses were built on top of the previously described bioinformatic methods in the previous example. Briefly after demultiplexing into individual barcodes, sequencing reads were aligned with either Bismark (a bowtie2 wrapper for bisulfite sequencing alignment) or BS-Bolt (a bwa-mem wrapper for bisulfite sequencing alignment). A lambda phage DNA construct with cytosine depleted PCR adapters spike in prior to bisulfite conversion was first examined to ensure high bisulfite conversion efficiency using sanger sequencing. Since this phage DNA is unmethylated, it was expected and confirmed that 99% of the cytosines were bisulfite converted.

After alignment, the CpG positions were extracted from the aligned reads and the CpG positions were binned based on genomic features such as H3K4Me3 histone marks for methylation dynamics validation. CpG positions can be extracted using either methylpy or the Bsbolt extraction method. The methylation frequency was then calculated as defined as the number of methylated CpG sites divided by the total number of CpG sites recorded in that window. The methylation frequency was then plotted across the features of interest. The detailed version of this protocol can be found in the supplementary methods

### Conclusion

Here, a new single cell WGBS sequencing method specific for the protocol provided herein was developed. methylated dCTPs in the gap filling step were used to protect the Tn5 adapter and cell barcode sequences from bisulfite conversion. In addition, a linear amplification step was includedas an attempt to recover the subset of unfragmented cDNA post bisulfite conversion. However, the yield of cDNA post bisulfite conversion was less than 1%. It was concluded that the cDNA library must be split from the DNA library or exponentially amplified prior to bisulfite conversion.

Then, a non-random priming post bisulfite conversion sequencing method was developed to efficiently extract the DNA from the gel beads via PCR and diffusion. The best post bisulfite adapter tagging method inspired by scnmC-seq which led to the creation of high complexity WGBS libraries was identified. To test the performance of the method, two single cell datasets were generated: an HCT116 cancer cell line dataset and a human kidney dataset. High bisulfite conversion efficiencies were validated using a lambda phage DNA spike in prior to bisulfite conversion and sanger sequencing. The analysis of the methylation levels over HCT116 H3K4Me3 histone marks recapitulated hypomethylation dynamics found in other studies (Sharifi-Zarchi et al. 2017). Furthermore, the kidney pilot study demonstrates the immense difficulty in performing single cell methylation analysis in terminally differentiated tissue. The paucity of CpG sites recovered at the sequencing cost per cell in this study prevents the discrimination of single cells. Since the number of recovered CpG sites is roughly 10 fold less than the number of CH sites. This low signal at high sequencing cost necessitates the need for RNA co-sequencing to assist in single cell clustering and cell type calling of terminally differentiated tissues.

Further optimizations at the single cell level of this method is described in the next Example. It is contemplated this method could readily be optimized for bulk WGBS. The higher alignment rates found in Tn5 based combinatorial indexing WGBS methods compared to single cell per well methods could be because of the efficient Tn5 insertion speculated previously (Mulqueen et al. 2018). The methods provided herein are thus a viable alternative to existing bulk WGBS methods.

### Example 4-3—Level Indexing Development and RNA Integration Part 2

Despite evidence of single cell resolution through the 2-level combinatorial indexing approach discussed above, the number of barcode collisions was inconsistently high. Published combinatorial indexing protocols typically have barcode collision rates less than 10% while the method described above had barcode collision rates between 15-40% (Mulqueen et al. 2021; 2018). The barcode collision rates are typically estimated by performing a human/mouse cell mixture experiment where equal numbers of human and mouse cells are mixed prior to the experiment. After sequencing, a mixed barcodes are identified as any barcode combination that contains at most 80% of reads from one species. The collision rate is then estimated as two times the mixed barcodes rate as doublets from the same species are not observed. Two sources of barcode collisions were identified: doublets that arise during the encapsulation process where two are more cells are captured by the same bead and two or more cells that have the same barcoding path. The latter factor is typically controlled by single cell sorting (Mulqueen et al. 2018; 2021). Because the gel beads are too large to be cell sorted by a typical FACS machine, estimating the concentration of cells prior to plating is required which leads to inherent inaccuracies that could cause barcode collisions. As a result, a third layer of combinatorial indexing was developed to scale the barcode space 100× and increase the tolerance of inaccurate cell number plating using these gel beads at dilute concentration. The increase in barcoding space has additional benefits. It expands the throughput of the technology 100×, vastly decreasing the number of experiments needed to characterize human tissues.

The co-sequencing of the transcriptome and the methylome is complicated by the bisulfite conversion process. Generally, multi-omic technologies have tackled this problem in two ways: separating the cDNA from the gDNA prior to bisulfite conversion or exponentially amplifying the cDNA with dmCTPs prior to bisulfite conversion. In scNMT-seq, single cells the RNA is separated from the gDNA with reverse transcription primers annealed to magnetic beads (Clark et al. 2018). In snmCAT-seq, full length cDNA is exponentially amplified with dmCTPs prior to bisulfite conversion. The cDNA is discriminated form the gDNA library after sequencing as the cDNA library is highly methylated compared to the DNA library. Previously, it was attempted to linearly amplify the cDNA with dmCTPs prior to bisulfite conversion. This resulted in an extremely low yield of cDNA libraries post bisulfite conversion. Here, an exponential cDNA amplification method prior to bisulfite conversion is explored like the snmCAT-seq design by designing a combinatorial barcoding approach without Tn5. However, the cDNA was observed to be too long to efficiently diffuse out of the gel bead. As a result, the cDNA need to be split prior to bisulfite conversion. Below are the solutions explored to arrive at this conclusion.

By combing two solutions: the splitting of cDNA prior to bisulfite conversion and drastically increasing the combinatorial barcoding space, the first workable solution was identified where the transcriptome and methylome are co-sequenced with doublet rate less than 10%. In addition, each enzymatic reaction was optimized to increase the library complexity of this workable solution over 100×. This solution utilizes an unstable encapsulation and gel formation process. Thus, a new solution that increases the consistency of the encapsulation process and subsequent library formation is provided.

### Methods and Results

### The Development of 3-Level Combinatorial Indexing

The cutting edge of combinatorial indexing technology development utilizes three or more levels of combinatorial indexing. This development crucially removes the need for cell or nuclei sorting to control barcode collision rates. There are three general methods for three-level combinatorial barcoding that have been demonstrated in single cell DNA accessibility and RNA technologies: 1) The use of Tn5 to insert the first barcode, ligation to the Tn5 overhang to add the second barcode, and PCR to add the third barcode; 2) The use barcoded reverse transcription primers to add the first barcode, ligation to the reverse transcription primer overhang to add the second barcode, and PCR to add the third barcode; and 3) The use of barcoded reverse transcription primers, linear polymerase-based extension to add the second barcode, and PCR to add the third barcode.

### Tagmentation Based 3-Level Indexing for WGBS

Three-level indexing using Tn5 based DNA accessibility sequencing or ATAC sequencing are at the cutting edge of combinatorial indexing technology. ATAC/RNA co-sequencing methods take advantage of the Tn5 overhanging sequences during Tn5 insertion to allow for a ligation of an additional barcoded adapter, increasing the combinatorial indexing level (C. Zhu et al. 2019; Domcke, Hill, Daza, Cao, O'Day, Pliner, Aldinger, Pokholok, Zhang, Milbank, Zager, Glass, Steemers, Doherty, Trapnel, et al. 2020; Plongthongkum et al. 2021).

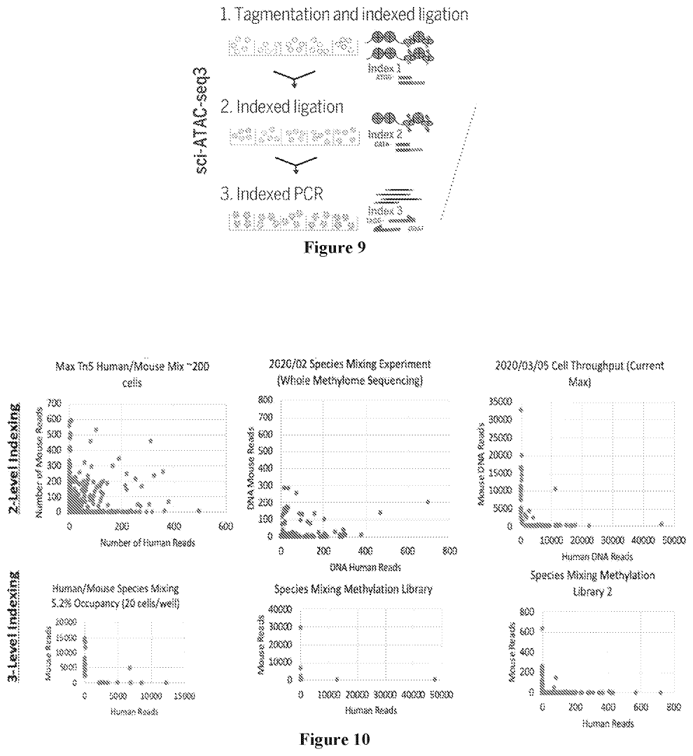

There are two major designs: the 3-level sci-ATAC design or the SPLIT-Seq design which is employed in methods such as SNARE-Seq2 and PAIRED-Seq. In the 3-level sci-ATAC design (FIG. 9), T7 ligase and a 15 bp synthetic splint oligo with a 3′ blocking modification to prevent extension by polymerases is used to ligate an adapter containing the second cell barcode. In the SPLIT-Seq design, T4 ligase and a 39 bp synthetic splint oligo is used to ligate an adapter containing the second cell specific barcode. The SNARE-Seq2 was SPLIT-Seq design was first attempted.

In summary, Tn5 is first used to insert the first cell barcode in the gel beads. Afterwards, T4 ligase is used to ligate the second cell barcode followed by PCR to add the third barcode using the gel bead platform. The qPCR results showed that the ligation was efficient as similar amplification dynamics between ligated and unligated templates were observed. PAGE also showed the shift in size owing to the ligation of adapters to the transposon overhang.

However, the design was not compatible with the WGBS design described herein. Sanger sequencing experiments revealed that one issue was the blunt-end ligation of mosaic end sequences. This prompted an attempt to try T7 ligase, which has no blunt-end ligation activity. However, a second problem was discovered: the splint oligo was blocking the gap filling step that is required for the WGBS design as discussed in the previous examples. The melting temperature of this splint oligo was too high (calculated to be 80° C.). In contrast, the mosaic end sequence melting temperature is 54° C. which allows the mosaic end to unanneal from the transposon sequence during the gap filling step which occurs at 72 C. One solution was to switch the polymerase from the high fidelity Q5 NEB polymerase to Taq polymerase as Taq polymerase can displace the splint oligo using a 5′ exonuclease capability. In contrast, Q5 polymerase does not contain any 5′ exonuclease or strand displacing capability. However, Taq polymerase was not compatible with the Tn5 fragmentation protocol. The first step in the gap filling protocol is to denature the Tn5. As previously published, this is typically performed using 0.1% SDS (Picelli, Björklund, et al. 2014). The SDS needed to be quenched with 2% Triton X prior to gap filling to prevent polymerase inactivation by SDS. Between Taq polymerase and Q5 polymerase, Q5 polymerase displays a much higher resistance to this denaturation and quenching protocol. Taq polymerase is inconsistently active during this protocol. This insight led to attempts to try the 3-level sci-ATAC ligation design.

The 3-level sci-ATAC design utilizes T7 ligase and, crucially, uses a shorter 15 bp splint oligo with a melting temperature of 58° C. This lower melting temperature allows for the splint oligo to easily unanneal from the adapter/transposon junction during gap filling which occurs at 72° C. FIG. 10 shows the success of this library construction with this method and consistent lower barcode collision rates between the 2-level indexing and 3-level indexing designs. This design shows incredible promise in the development of both a single cell whole genome sequencing and whole genome bisulfite sequencing method at the scale of tens of thousands of cells per experiment with just three 96 well plates.

The detailed protocol to generate these libraries is described below. Briefly, the encapsulated beads are first split into a 96 well plate containing 100-200 encapsulated beads per well. Following the previous 2-level indexing protocol, the beads are tagmented with Tn5 adding the first cell barcode. The beads are then pooled, washed, and split into a second 96 well plate where the second cell barcode is ligated onto the Tn5 sticky end. The beads are then pooled and then split again to a third 96 well plate where roughly 40 encapsulated cells or nuclei are input per well. In the case of whole genome sequencing, PCR primers are added after Tn5 fragmentation to amplify the library and add the third cell barcode. In the case of whole methylome sequencing, the same protocol described in the previous example is performed but the linear amplification barcoded primer after bisulfite conversion is reverse complement to the ligated adapter.

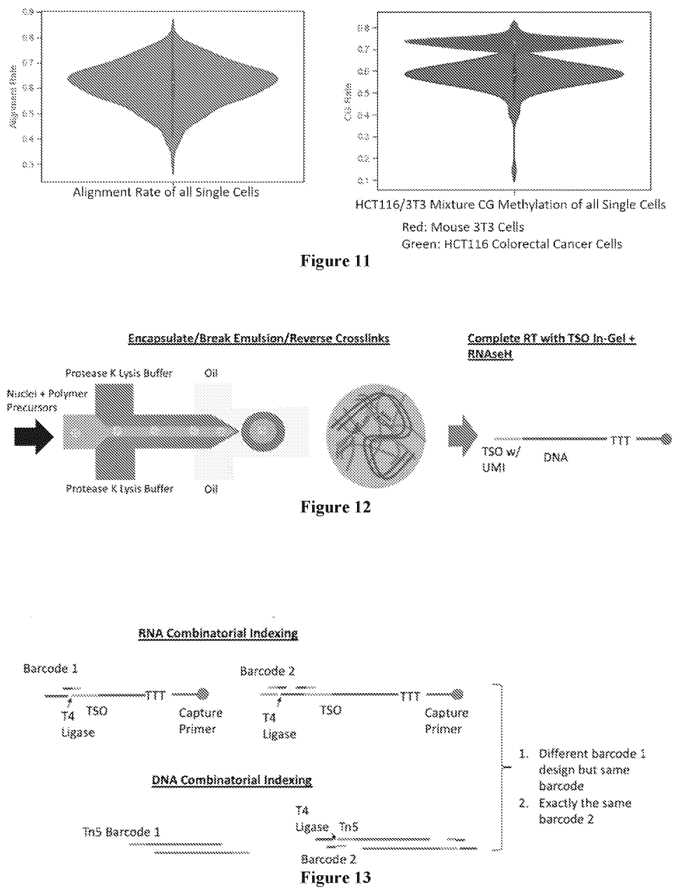

FIG. 11 shows the sequencing statistics at the single cell level using the 3-level combinatorial indexing method. The method demonstrates high alignment rates, a mean alignment rate of 62+/−8.4%, like the previous 2-level indexing method. Furthermore, it is shown how the global CG methylation could be used to discriminate single cells in a cell mixture of human cancer HCT116 colorectal cells and mouse fibroblast 3T3 cells. The hypomethylation of HCT116 cancer cells compared to non-cancerous tissue has been described in previous studies (Lengauer, Kinzler, and Vogelstein 1997).

The Development of the cDNA Recovery Method
Exponentially Amplifying cDNA Prior to Bisulfite Conversion

From the previous examples, the remaining major challenge of the incorporation of cDNA co-sequencing with WGBS. Building on previous observations therein, it was reasoned that exponentially amplifying cDNA prior to bisulfite conversion could generate enough cDNA product that is recoverable post bisulfite conversion fragmentation. This was inspired by scnmCAT-seq where 10 cycles of full-length cDNA amplification prior to bisulfite conversion resulted in enough intact cDNA fragments to recover the transcriptomes of single cells (Luo et al. 2022). The challenge was to first generate full-length cDNA in the gel bead platform. The exponential amplification of cDNA as demonstrated in SPLIT-Seq, SNARE-Seq2, and PAIRED-Seq relies on the addition of a template switch oligo (TSO) once reverse transcriptase reaches the 5′ end of the RNA. This takes advantage of a feature of reverse transcriptase to often add cytosines on the extended cDNA product. This is beautifully demonstrated in the single cell sequencing technique, SMART-Seq (Picelli, Faridani, et al. 2014). Typically, reverse transcription with the addition of a TSO requires 90 minutes of incubation at 42° C. to complete. In addition, the RNA needs to be free of RNA binding proteins for the reverse transcriptase to reach the 5′ end of the RNA. Together, this requires the nucleus to sufficiently denatured. Thus, combinatorial indexing methods that use the nucleus like SPLIT-Seq require two rounds of reverse transcription. The first round is a partial reverse transcription that ensures that the RNA is binding to the reverse transcription primer is stable. After combinatorial indexing, the nucleus is denatured along with the RNA binding proteins. A second round of reverse transcription is required for the cDNA to be extended to the 5′ end of the RNA and template switching to occur (Rosenberg et al., n.d.; C. Zhu et al. 2019; Plongthongkum et al. 2021). Secondly, a barcoding scheme was developed that protected the cDNA from Tn5 barcoding (necessary for the barcoding of gDNA for WGBS). Tn5 barcoding would fragment the cDNA and prevent the exponential amplification of full-length cDNA using the TSO and capture primer PCR adapter sequences.

In the gel bead platform, partial reverse transcription prior to in-nuclei encapsulation was performed. After nuclear and RNA binding protein denaturation inside of the gel bead, reverse transcription is then completed with a TSO in a similar fashion with a few modifications. TSO based reverse transcription in polyacrylamide gel beads was first documented in a single cell RNA sequencing polyacrylamide gel bead protocol called BAG-Seq (Li et al. 2020). Instead of the typical 42° C. for 90 minutes reverse transcription, this protocol utilizes 42° C. for 60 minutes followed by 50° C. for 60 minutes to account for reverse transcriptase and TSO diffusion through the gel bead. Utilizing this reverse transcription protocol, full length cDNA was created with the capture primer adapter on one end and TSO adapter on the other.

The next challenge was to develop a barcoding scheme that allows for the full length cDNA amplification prior to bisulfite conversion. Previously published cDNA combinatorial indexing methods leverage the 5′ overhang on the capture primer to ligate cell barcode adapters (Rosenberg et al., n.d.; C. Zhu et al. 2019; Plongthongkum et al. 2021). Because the 5′ end of the capture primer in this method is modified with an acrydite, this ligation protocol cannot be used. However, a 3′ cDNA overhang could be created if the RNA and TSO sequence could be removed as shown in FIG. 12. The RNA can be digested with RNAseH and the TSO sequence could either also be digested with RNAseH or with brief high temperature heating and blocking with a sequence reverse complement to the TSO to prevent the TSO from reannealing to the single stranded cDNA.

With the removal of RNA and the TSO, the DNA was first tagmented to insert the first DNA barcode as described previously. The cDNA is not tagmented because it is single stranded. In the same well, w an adapter was then ligated to the TSO end of the cDNA. Although the DNA and cDNA barcode designs are different, the barcode itself is the same. The beads are then pooled, split into a second 96 well plate and T7 ligation performed with the same adapter as described previously in the 3-level WGBS method. FIG. 13 illustrates this method.

This approach was observed to be a more inefficient way to extract cDNA as the amount of diffusion out of the gel bead was too low to consistently generate high quality libraries. Thus, a Tn5 based approach was reverted to in order to fragment the cDNA and allow sufficient extraction of these sequencing from the gel bead. Furthermore, the amplification of ligated TSO products produced mostly off-target products. This could be due to the non-specificity of the addition of the TSO sequence during reverse transcription.

3-Level Tagmentation-Based cDNA Generation Protocol

With the exponential amplification of cDNA deemed an inviable approach, it was decided to split the cDNA library and gDNA prior to bisulfite conversion. In this approach, full length encapsulated cDNA inside of the gel bead after encapsulation were created like previously described but without the TSO sequence. A protocol was then to perform second strand synthesis of the cDNA using a mixture of RNAseH, DNA polymerase I, and DNA ligase slowly degrade the RNA and create a second strand of DNA complement to the one synthesized during reverse transcription.

This double stranded cDNA and DNA are then tagmented with the same barcode followed by ligation with the same barcoded adapters. Prior to bisulfite conversion, the cDNA was then linearly amplified for 10 cycles as described previously with a few modifications. Firstly, the linear amplification PCR reaction volume was doubled. After linear amplification, each reaction was pelleted at 300 g for 2 minutes and vortexed to resuspend the beads twice. This was used to assist in the diffusion of linearly amplified products from the gel beads. Finally, the beads were pelleted, and half of the supernatant was carefully removed without disturbing the bead pellet and transferred into a separate plate. It was found that this is crucial as the majority of the gDNA is still inside of the gel bead. Thus, the majority of the gDNA is in the original plate containing the beads while a fraction of linearly amplified cDNA is separated into the separate plate. After splitting the libraries, bisulfite conversion reagent is added to the gDNA plate, and WGBS library construction proceeds as previously described. In the separated cDNA plate, barcoded primers reverse complement to the ligation adapter is added and 7 cycles of exponential amplification are performed. SPRI bead purification was then performed using a 0.8×ratio of each well followed by a second round of exponential amplification with PCR primers containing Illumina sequencing adapters. After this PCR is complete, the libraries were pooled followed by two rounds of SPRI bead purification using a 0.8×ratio to prepare the library for sequencing.

To test the success of the combinatorial barcoding scheme, libraries from a cell line mixture of human and mouse cells like described previously were generated. The resulting DNA library had low barcode collision rates. In contrast, the RNA library was completely mixed. It was hypothesized that this mixing result was because the method of cDNA synthesis prior to combinatorial indexing generated too much background which would be covalently attached to the gel beads during encapsulation. In contrast, other combinatorial indexing approaches that perform in-nuclei cDNA generation gradually remove the background cDNA as the nuclei are washed between each combinatorial indexing step. Most of this background occurs during the nuclei isolation as the cytoplasmic RNA can remain after cell lysis. In addition, the standard pelleting and washing of nuclei during these steps typically result in extensive nuclei lysis. Thus, it was predict that the success of the method relies on high quality nuclei/cell isolation techniques that minimize background RNA.

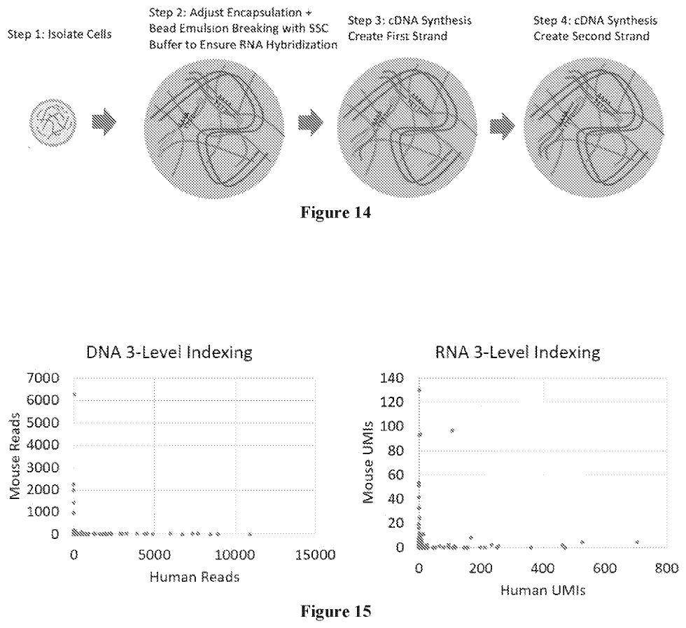

To minimize background RNA, it was decided to first develop a new protocol using single cells. As mentioned previously, cell lysis during nuclei isolation generates extensive free RNA which could be covalently attached to the gel beads causing extensive barcode collisions. Instead of performing in-situ reverse transcription, it was decided to first encapsulate and lyse the cells with a key modification. The cells would be co-encapsulated with the acrydite modified reverse transcription primers to allow for the capture of RNA polyadenylated bases. The emulsion breaking buffers were modified to include saline-sodium citrate buffer (commonly known as SSC buffer). This high salt buffer enhances the stability of the polyadenylated and reverse transcription primer hybridization to prevent the free diffusion of RNA after encapsulation. Full length cDNA is then generated as described previously in the gel bead. FIG. 14 illustrates this protocol.

Libraries from a cell line mixture of human and mouse cells were then generated using the same protocol described previously to assess the barcode collision rates. FIG. 15 shows the success of this method where both DNA and RNA libraries demonstrate low barcode collision rates.

### Biological Validation of RNA Libraries

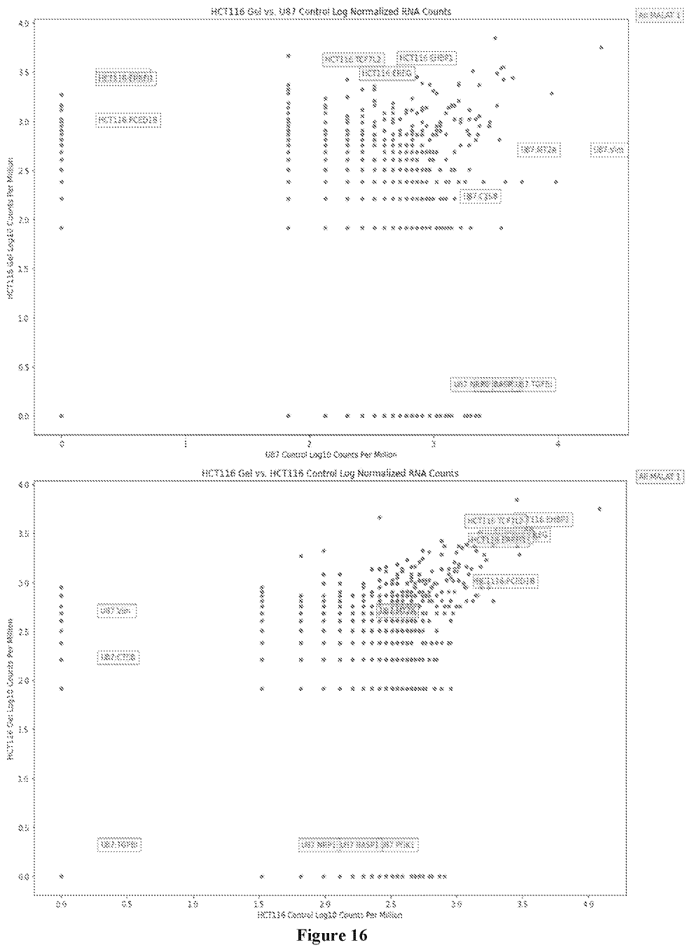

After identifying the correct RNA sequencing strategy, the biological relevance of the libraries was assessed. Three RNA libraries using the method were created: encapsulated HCT116, in-tube HCT116, and in-tube neuroblastoma U87 cells. After sequencing, the gene counts of each library were correlated, and marker genes were identified. Briefly, the single cell resolution encapsulated HCT116 library were first bulked to enhance correlations. The cDNA reads were trimmed, filtered, and then aligned to the human genome using STAR. The htseq package was then used to generate a gene counts matrix. The gene counts matrix was then log normalized using scanpy. Log normalized counts per million of the in-tube HCT116, in-tube U87, and encapsulated HCT116 were then plotted. Marker genes for HCT116 and U87 found in literature were then labeled. The details of how this analysis was performed is documented in the supplementary methods. FIG. 16 shows that the gel encapsulation HCT116 RNA sequencing technique recovered the expected marker gene expression. Highly expressed marker genes for the neuroblastoma cells such as Vim are only expressed in brain tissue. The low expression of these gene among other U87 marker genes found in the HCT116 libraries validated the biological relevance of the RNA sequencing method.

### Optimizations of Library Formation and Performance

After demonstrating the potential of the 3-level WGBS and RNA co-sequencing method, it was necessary to assess the consistency of the method and library complexity. The table below illustrates the variability in barcode collision rates across various experiments. Published combinatorial indexing methods typically result barcode collision rates no more than 10% (Rosenberg et al., n.d.; Plongthongkum et al. 2021; C. Zhu et al. 2019; Mulqueen et al. 2018).

| Experiment Number | DNA collision rate |
|---|---|
| 1 (first L3 experiment | 6% |
| 2 | 4% |
| 3 (New lysis buffer) | 8% |
| 4 | 40% |
| 5 | 8% |
| 6 | 11% |
| 7 | 30% |
| 8 | 15% |

Thus, the potential causes of this variability was then explored. Across multiple encapsulations, an imaging analysis to correlate potential features with high barcode collision rates was performed It was observed that the freeze/thawing process that is typically employed to store the beads to preserve RNA integrity prior to reverse transcription caused extensive aggregation and gel bead destruction.

Encapsulation quality variability which was determined to be caused by two factors: 1) the hydrophobic coating of the microfluidic device and 2) the polymerization of the gel prior to encapsulation. Inconsistent bead sizes due to the unoptimized hydrophobic coating of the microfluidic device and the non-spherical gel bead products that result from the partial polymerization of polyacrylamide prior to encapsulation were observed.

To address the variability of bead sizes, it was noticed that the droplet formation on the microfluidic device was inconsistent. The presence of larger than designed bead sizes leads to the increased probability of multiple cells or nuclei encapsulated in the same bead and heightened barcode collision rates. Originally, the microfluidic hydrophobic coating method described in inDrops was used (Klein et al. 2015). Briefly, the device is coated with aquapel, air-dried, coated with FC-40, and then air dried. It was observed that FC-40 did not dry easily, and residual FC-40 prevented the proper formation of droplets. An aquapel coating was developed with, air dying, coating with isopropyl alcohol, followed by device drying at 55° C. for 30 minutes. The isopropyl alcohol maintains the hydrophobicity of the microfluidic device while drying more easily than FC-40.

During the development of the RNA co-sequencing method, the microfluidic BAG-seq encapsulation scheme was adapted to capture the RNA summarized in FIG. 17. Interestingly, the polymerization initiator, ammonium persulfate (APS), was mixed the polymer precursor. In these experiments, it was found that this encapsulation scheme resulted in gradual polymerization of the acrylamide prior to encapsulation. Thus, the non-spherical beads are the result of non-uniform polymerization of the acrylamide. In addition, this polymerization prior to encapsulation results in cells simply embedded into the gel instead of lysed and uniformly immobilized by the gel bead matrix. These poorly immobilized DNA and cDNA would cause extensive mixing resulting in elevated barcoded collision rates.

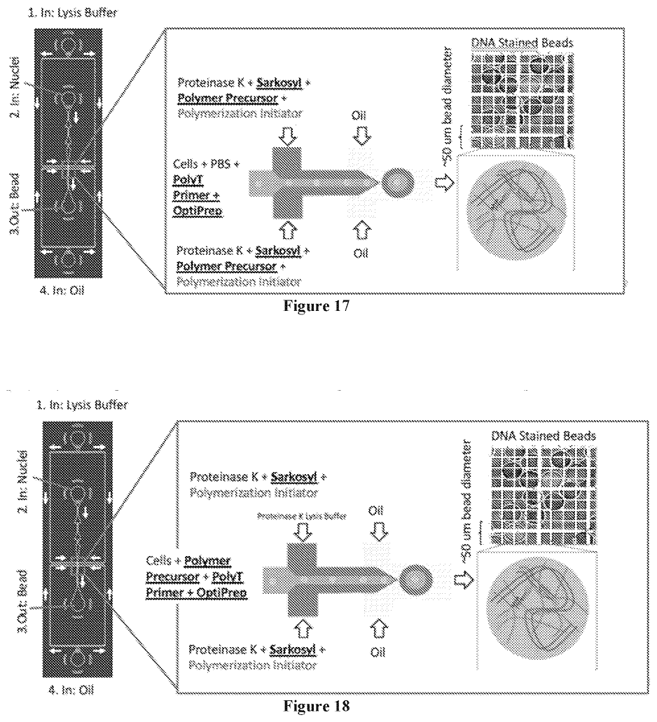

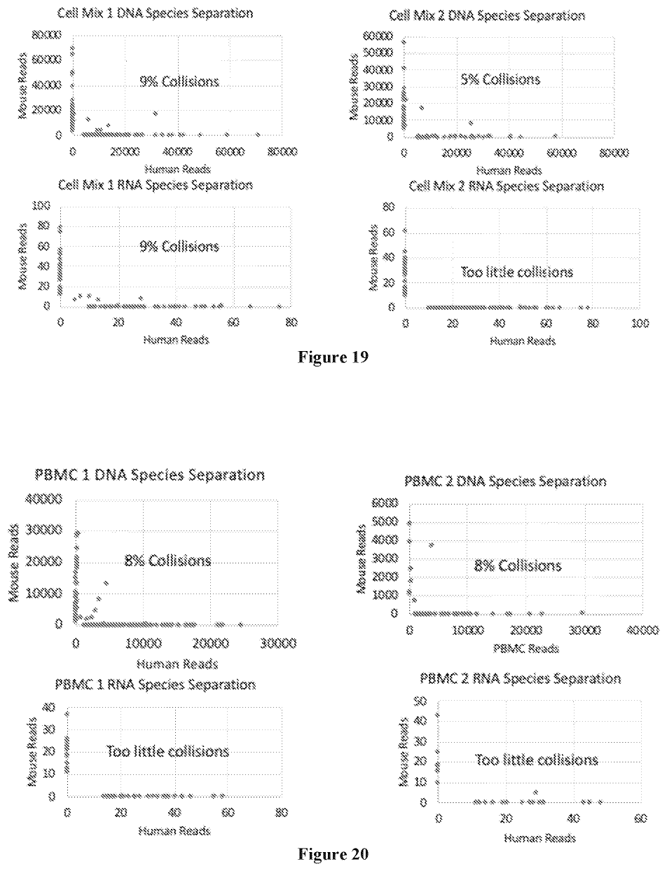

Thus, the polymer precursors and APS were separated in the encapsulation scheme illustrated in FIG. 18. Interestingly, this resulted in poor lysis quality. Thus, reoptimization of the lysis detergents (to 0.5% SDS) to ensure high quality lysis and uniform entanglement of DNA within the polyacrylamide gel matrix was necessary. The results of these experiments is illustrated in FIG. 19.

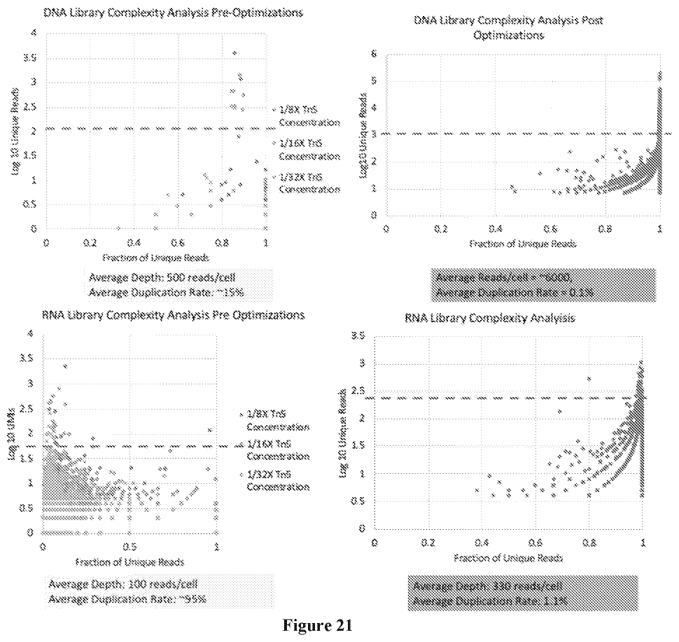

Next, the robustness of the encapsulation method with a human peripheral blood mononuclear cell (PBMC) mixture was assessed. To ensure high quality live cells, a dead cell magnetic separation technique was first performed. FIG. 20 shows the success of the encapsulation protocol in two PBMC samples.

With the improved encapsulation stability and consistent low barcode collision rates, improving the library complexity was attempted. Briefly, each barcoding reaction was optimized: 1) the Tn5 insertion reaction, 2) the ligation reaction, and 3) the post bisulfite tagging and PCR reactions. d Tn5 reaction concentrations were screened starting at 0.05 mg/mL and identified the optimal Tn5 concentration for 100-200 encapsulated cells to be 0.00625 mg/mL. The optimal reaction time was found to be 90 minutes. The optimal T7 ligase concentration was 0.75 U/μL (2.5×higher than standard reaction conditions). Ligation times did not increase library complexity. It was observed that it was crucial for each well in the final PCR plate to be processed individually even after barcoding was complete. The exponential amplification of each well prior to pooling minimizes stochiometric barcode path biases that are commonly observed given the hundreds of barcoding reactions in the protocol. FIG. 21 shows the results of these optimizations at the single cell level. The combination of these optimizations resulted in at least 100× increase in library complexities. For downstream single cell RNA analysis, previous publications used a 200 genes per cell cut-off. For downstream single cell DNA methylation analyses, sciMET uses a 30,000 unique reads per cell cut-off. From these results, it is conservatively estimate that the method could detect at least 1000 genes per cell and over 100,000 unique WGBS reads. These library complexity metrics give promising preliminary evidence that the method could be used for human tissue profiling.

### Conclusion

Described herein is the culmination of foundational works described in the previous examples resulting in a successful prototype 3-level WGBS and RNA co-sequencing method. There are currently no methods with the same throughput and co-sequencing capabilities as the one described here. Expanded upon were previous 2-level combinatorial indexing protocol to solve two major problems: inconsistent barcode collision rates and loss of the cDNA library. To make the leap from a 2-levels to 3 levels of combinatorial indexing, two different barcode ligation paradigms were tested: SPLIT-seq T4 ligation and 3-level sci-ATAC T7 ligation (C. Zhu et al. 2019; Domcke, Hill, Daza, Cao, O'Day, Pliner, Aldinger, Pokholok, Zhang, Milbank, Zager, Glass, Steemers, Doherty, Trapnel, et al. 2020; Rosenberg et al., n.d.; Plongthongkum et al. 2021). Herein, the positive and negative aspects of each approach were and explained the reasoning behind choosing the 3-level sci-ATAC T7 ligation approach.

Also explored herein were two potential solutions to the loss of cDNA: splitting the cDNA library prior to bisulfite conversion and exponentially amplifying it. This led to the adaptation of a wide swath of single cell RNA sequencing methodologies: the partial in-nuclei reverse transcription adapted from SPLIT-Seq followed by the SMART-Seq2 full-length cDNA amplification and coupled with the scmCAT-Seq adaptation for bisulfite conversion and the sci-RNA cDNA tagmentation based approach. It was found that the full-length cDNA exponential amplification approach could not sufficiently generate enough cDNA to diffuse out of the gel beads for sequencing. The most promising approach was to use Tn5 to sufficiently fragment the cDNA to allow for diffusion during the linear amplification step prior to bisulfite conversion.

The cDNA and gDNA libraries were then split prior to bisulfite conversion. However, it was found that the partial in-nuclei reverse transcription approach created too much free cDNA background that eventually was covalently attached to the polyacrylamide beads after encapsulation. Thus, this background caused extensive barcode collision rates. The most promising solution by performing reverse transcription and second strand synthesis in gel was then identified. This approach combined with the cDNA splitting approach successfully created both DNA and cDNA libraries with low barcode collision rates. The Table below summarizes the single cell RNA sequencing methods adapted and tested in the gel bead platform that guided the development of the correct method. The lessons learned from each preceding examples in addition to the ones described here culminated in a final working prototype that is graphically summarized in FIG. 1, which depicts an optimized process overview according to the methods provided herein.

| | RNA Extracted/Confirmed | |
|---|---|---|
| Method Adapted to Gel Beads | by Sequencing | Single Cell? |
| Sci-RNA seq | 1% alignment rate | ~50% doublet rate |
| SPLiT seq/SNAR2-seq/PAIRED-seq | cDNA too long to diffuse | |
| | out of gel bead | |
| SPLiTseq/SNAR2-seq/PAIRED-seq + | 50% alignment rate | 100% doublet rate |
| Sci-ATAC-3-level | | |
| In-Gel RT + sci-ATAC 3-Level | 48% | 8.60% |

The protocol provided herein was further optimized to resolve inconstancies in the polyacrylamide gel bead formation and performed a human tissue a proof of concept with PBMCs. The optimizations of each barcoding reaction that led to over 100× increase in library complexity compared to the initial prototype. The specific protocol described herein can process 50,000-100,000 cells per experiment with three 96 well plates. With further optimization using 384 well plates could increase the throughput of this platform to 3,000,000-5,000,000 cells per experiment which could be used to profile organ systems. Future work involving the methylome profiling of the PBMCs would showcase the capabilities of this method and be the first multi-omic RNA and DNA methylation study of PBMCs at the single cell level. Furthermore, this work would demonstrate the ultra-high throughput capabilities of the technology. Specifically, the single cell RNA datasets of the PBMC sample could be projected onto the 10×PBMC reference dataset using Seurat. Cell type labels from this reference could be transferred to the single cell RNA datasets to assist in cell type calling and the formation of pseudo bulk methylomes. As mentioned previously, the creation of pseudo bulk methylomes could generate enough methylome coverage for the identification of cell-type specific differentially methylated regions using CG methylation in PBMCs that have never been profiled at the cell-type level. Careful optimization of nuclei isolation methods to minimize cell free RNA could also enable the use of nuclei with this method. The ability to process nuclei would allow this protocol and pseudo bulk methylome analysis framework to add cell type specific methylation annotations to recently published single cell RNA atlases of terminally differentiated tissues such as human kidney and lung (Travaglini et al. 2020; Lake et al., n.d.).

### Example 5—Supplemental Methods

The experiments and methods described above were performed according to the following general protocols. It is contemplated that deviations from the methods provided below could be performed consistent with the instant disclosure.

### Summary of the Optimized 3-Level Combinatorial Indexed Co-Sequencing Method

The foundation of the platform is the encapsulation of single cells containing lysis buffer and acrylamide monomer in an oil emulsion using a microfluidic device droplet maker (e.g., those as used by 10× Genomics). Reverse transcription primers have 5′ acrydite modifications to co-polmyerize with the acrylamide and capture the RNA. After an overnight incubation, each droplet polymerizes into a polyacrylamide bead with the genomic DNA dispersed and intertwined in the polyacrylamide matrix. The acrydite group incorporates the reverse transcription primers to the polyacrylamide back bone. The RNA hybridizes to the reverse transcription primers and are anchored to the gel bead. This polyacrylamide gel bead is accessible to the enzymes critically responsible for cDNA synthesis and combinatorial barcoding. After emulsion breaking, the beads undergo reverse transcription as described in other studies and second strand synthesis overnight (Li et al. 2020).

The DNA and RNA barcoding scheme is like previously published Tn5 based split and pool combinatorial barcoding methods but adapted for polyacrylamide beads as opposed to nuclei (Domcke, Hill, Daza, Cao, O'Day, Pliner, Aldinger, Pokholok, Zhang, Milbank, Zager, Glass, Steemers, Doherty, Trapnell, et al. 2020; Cao et al. 2019). Briefly, the beads are dispersed into a 96 well plate so that each well contains roughly 200 encapsulated cells or nuclei. Hyperactive Tn5 containing 5′ phosphorylated transposons tagment the beads adding the first DNA barcode using optimized reaction conditions found in this work. The beads are then pooled, washed, and split into a second 96 well plate where the second DNA barcode is ligated to the transposon overhang. Finally, the beads are then pooled, washed, and split into a third 96 well plate. Linear amplification is used for 10 cycles to first amplify the cDNA allowing it to diffuse out of the gel bead to split the cDNA libraries from the gDNA using a PCR primer reverse complement to the reverse transcription primer sequence. The beads are then pelleted and 50% of the supernatant containing the cDNA is exponentially amplified for 7 cycles adding the third barcode to the cDNA. The cDNA reaction is then bead purified using SPRI beads at a 0.8×ratio followed by another 10 cycles of PCR using a P5 primer and an i7 primer. Once this reaction is complete, the wells are pooled and 0.8×bead purification was performed twice on the pool.

After linear amplification and extraction of cDNA, the gDNA bisulfite conversion reagent is added to the remaining gDNA for bisulfite conversion. The manufacturers protocol for desulphonation is followed with a key modification. At this point, the magnetic beads coat the gel beads which contain the gDNA. Instead of eluting the DNA from the magnetic beads, the magnetic beads were taken along with the gel beads and added them to a PCR reaction where the gDNA is linearly amplified for 20 cycles with primers hybridizing to the ligated adapter. This process allows gDNA to diffuse out of the gel bead. The third barcode is added to the gDNA during this linear amplification process. rSAP is then added to the reaction to remove all 5′ phosphates that could potentially interfere with the adaptase protocol. The DNA is then bead purified using SPRI beads at a 1.2×ratio and eluted into the standard adaptase reaction protocol, following the manufacturer's instructions. PCR master mix containing a P5 primer and an i7 primer is then added to the heat inactivated adaptase reaction as described in scnmC-seq (Luo et al. 2018). 8 cycles of exponential amplification are then performed. The reaction was then bead purified at a 0.8×ratio followed by another 8 cycles of PCR using P5 and P7 primers. Finally, the wells are pooled and 0.8×bead purification was performed twice on the pool.

Single-Cell 3-Level Detailed mDNA/RNA Gel Bead Sequencing Materials

### Equipment

| | | Catalog |
|---|---|---|
| Name | Company | Number |
| Inverted microscope | Fisher Scientific | 29AX |
| | Education | |
| 3 mL syringes | Beckton Dickinson | 309657 |
| PE-50 tubing | Instech | BTPE-50 |
| Leur stub 22 ga | Instech | LS22 |
| Right angle couplers 22 ga | Instech | SC22/15RA |
| PDMS Co-Flow Microfluidic | | |
| Droplet Device | | |
| 30 mm cell strainer | Fischer Scientific | NC9682496 |
| Deep well magnetic plate | Thistle Scientific | VP 771HH-LF |

### Chemical Reagents/Solutions

| Name | Company | Catalog Number |
|---|---|---|
| Nuclei Isolation | | |
| Nuclei Isolation Buffer (10 | — | — |
| mM Tris-HCl, 10 mM NaCl, | | |
| 3 mM MgCl2, 0.1% Igepal) | | |
| Bovine Albumin Fraction V | Fischer Scientific | 15260037 |
| (7.5% solution) | | |
| SUPERase•In ™ RNase | Thermo Fisher Scientific | AM2694 |
| Inhibitor (20 U/μL) | | |
| Aqueous Phase 2 | | |
| Acrylamide | Fisher BioReagents | BP170-500 |
| Bisacrylamide | Sigma-Aldrich | M729-100G |
| 0.1M DTT | Thermo Fisher Scientific | 707265ML |
| Ammonium Persulfate | Sigma-Aldrich | 09913-100G |
| Sodium dodecyl sulfate | Thermo Fisher Scientific | 28364 |
| (SDS) | | |
| Proteinase K | NEB | P8107S |
| Oil Phase | | |
| HFE-7500 (3M) | Oakwood Chemical | 051243 |
| TEMED | Sigma-Aldrich | 1.10732 |
| 008-FluoroSurfactant | Ran Biotechnologies | 008-FluoroSurfactant-5G |
| Aqueous Phase 1 | | |
| Nuclei Buffer (10 mM Tris- | — | — |
| HCl, 10 mM NaCl, 3 mM | | |
| MgCl2) or PBS for cells | | |
| OptiPrep | Stem Cell Technologies | 7820 |
| Nuclei or Cells | — | — |
| Post-Droplet Generation | | |
| Mineral Oil | Sigma-Aldrich | 69794-500ML |
| 1H,1H,2H,2H- | Synquest Laboratories | 2101-3-20 |
| Perfluorooctan-1-ol (PFO) | | |
| Tris-Tween Buffer: 100 mM | | |
| Tris-HCl pH = 8.0, 0.1% v/v | | |
| Tween 20 | | |
| SYTO ™ Green Fluorescent | | |
| Nucleic Acid Stain (300 uM) | | |
| Combinatorial Indexing | | |
| Transposon Annealing | — | — |
| Buffer: 400 mM Tris-HCl, | | |
| 500 mM NaCl | | |
| Diagenode Tagmentase | Diagenode | C01070010-20 |
| 2X Tagmentation Buffer: | — | — |
| 66 mM Tris-Acetate, 132 mM | | |
| K-Acetate, 20 mM Mg- | | |
| Acetate, 32% DMF | | |
| 2X T7 DNA Ligase Reaction | NEB | M0318L |
| Buffer | | |
| T7 Ligase | NEB | M0318L |
| 0.3% SDS | — | — |
| 10% Triton-X | — | — |
| Q5 ® High-Fidelity DNA | NEB | M0491L |
| Polymerase | | |
| 5-methyl-dCTP | NEB | N0356S |
| Advantage ® UltraPure dNTP | ClonTech | 639132 |
| Combination Kit | | |
| EZ-96 DNA Methylation- | Zymo | D5044 |
| Direct MagPrep | | |
| Post Bisulfite Conversion | | |
| KAPA HiFi HotStart Uracil + | KAPA/Roche | KK2802/07959079001 |
| Kit (250 rxn) | | |
| SPRISELECT, 60 ML - | Beckman Coulter | B23318 |
| Kapa Hifi Hotstart | Roche | 7958935001 |
| ReadyMix, KK2602, 6.25 ml | | |
| xGen ™ Adaptase ™ Module | IDT | 10009826 |
| 96 rxn | | |
| Shrimp Alkaline Phosphatase | NEB | M0371L |
| (rSAP) | | |

Single-Cell 3-Level Detailed mDNA/RNA Gel Bead Library Preparation

### Microfluidic Device Fabrication

### Creation of the Microfluidic Device Mold

The creation of the microfluidic device mold follows some standard SU-8 photolithography and microfabrication techniques. The process used was that described in the thesis of Andrew Richards discussed supra (scholarship.org/uc/item/4zk292pm).

This process wholly occurs inside a clean room as ambient dust particles could interfere with the microfluidic device feature formation. Briefly, 4-inch test grad silicon wafers were carefully rinsed with Piranha solution followed by rinsing in acetone, isopropyl alcohol, and finally DI water. This process is required to remove any organic residues off the wafer to ensure stability of the mold. The wafter was then blow-dried with nitrogen. The wafer was then cleaned by oxygen plasma at 5 sccm O₂ with 250 W power for 5 minutes using a PE-Etch 100. Su-8 2025 was then spin coated at 500 RPM for 10 seconds accelerated at 100 RPM/second followed by 3000 rpm for 30 seconds accelerated at 300 RPM/second. The wafer was then soft baked at 65 C for 2 minutes followed by 95 C for 5 minutes. The wafer was then UV-exposed using an EVG 620 mask aligner with a custom photomask. The wafer was exposed in hard contact mode for 12.3 seconds for a total exposure of 160 mJ/cm2. The custom photomask was ordered from a commercial vendor (FrontRange PhotoMask) with 10 micron tolerance, dark field background, and right read (chrome) down. The wafer was then carefully post exposure baked at 65 C for 1 minute followed by 95 C for 5 minutes. Afterwards, the wafers were developed in SU-8 developer by steady agitation until the features appeared. The wafer was periodically rinsed with isopropyl alcohol to check for the presence of unpolymerized SU-8.

Undeveloped SU-8 leaves a clearly white residue on the wafer. Continual exposure to the SU-8 developer will eventually remove all the white residue upon exposure to isopropyl alcohol.

After the features are clearly seen and no white residue is detected upon rinsing with isopropyl alcohol, the wafer was blow dried with nitrogen. The wafers were then hard baked at 150 C for 5 minutes to increase the thermal stability of the features. The wafers were then silanized using fluoro-octyl-trichloro-silane to allow for PDMS stamping using a vacuum chamber for 30 minutes of vapor deposition.

### Creation of the Microfluidic Device

The wafers were then transferred to 15 cm petri dishes and ˜80 g of PDMS mixed with 10% crosslinker was then cast onto the wafer inside the petri-dish, covering the features of the mold. Roughly 10 g of PDMS are then added to two 10 cm dishes, covering the bottom surface. The PDMS was then degassed by placing it inside of a vacuum chamber for 5 minutes, relieving the pressure and popping the bubbles with nitrogen gas, and repeating the process twice. The PDMS coated 10 cm dishes and mold was then polymerized at 80 C for 1 hour. Using an Exacto knife, two devices were cut from a single mold. Subsequent casting requires much less PDMS (roughly 20 g of PDMS with 10% crosslinker) just enough to cover the cut-out devices. The inlets/outlets were individually bored out with a 0.75 mm biopsy hole punch. 3M tape was then placed on the devices and then removed twice to remove PDMS debris from the microfluidic features. Next, the PDMS devices and 10 cm dishes were then plasma activated with a PE-Etch 100 by placing the devices on the middle rack exposed to 250 W power with 5 sccm 02 for 15 seconds. The bottom of the device was then quickly bonded to the coated 10 cm dishes after plasma activation and lightly pressed to encourage plasma bonding. The plasma bonded microfluidic devices were then baked at 80 C for 20 minutes to finish the bonding process and ensure stability of the bond.

### Hydrophobic Coating of the Microfluidic Device

For droplet formation during microfluidic encapsulation to occur, the microfluidic device must be coated with a hydrophobic coating. Aquapel is first filtered through a 30-micron filter to remove dust and precipitates. Using a P20 pipette, carefully pipette aquapel through each of the devices to uniformly coat all the features and incubate for at least 1 minute. Air was then used to push out the aquapel. This was done with a syringe or lab air valve attached to a pipette tip or microfluidic adapter. The device was then washed once with isopropyl alcohol by similarly pipetting it through each of the channels and then pushed out with air similarly as with the aquapel coating. Finally, the microfluidic devices are then dried in a 55 C incubator for 30 minutes.

### Cell Encapsulation

The protocol for performing the cell encapsulation of the optimized methods provided herein is performed according to the process outlined as follows: 1) Trypsonize cells and wash once with 1×PBS by pelleting cells at 300×g for 00:04:00. 2) Resuspend cells in 3000 cells/uL in encapsulation buffer: 1×PBS, 40% OptiPrep, 0.75% BSA, 5 μM reverse transcription primer, 1% v/v SUPERase RNAseInhibitor. 3) Create polyacrylamide buffer. In the formula below, the resulting polymer has a 0.9% crosslink percentage.

| Polyacrylamide Buffer | Volume (μL)/Mass | | Final Concentration | |
|---|---|---|---|---|
| 4M Tris-HCl pH = 7.5 | 175 | | 70 | mM |
| Acrylamide | 4.5 | g | 45% | w/v |
| Bisacrylamide | 44 | mg | 0.44% | w/v |

| | |
|---|---|
| H₂O | 9825 |

4) Create lysis buffer:

| Aqueous Phase 2 Solution | Volume (uL) | Final Concentration | |
|---|---|---|---|
| Polyacrylaide Buffer | 447.5 | — | |
| 20% w/v Sarkosyl | 5 | 0.2% | |
| 0.1M DTT | 15 | 3 | mM |
| Igepal | 5 | 1% | |
| Ammonium Persulfate | 12.5 | 0.5% | |
| (20% w/v) | | | |
| Proteinase K (0.8 U/uL) | 10 | 0.016 | U/uL |

| | | |
|---|---|---|
| Total | 500 | — |

5) Create oil solution:

| Oil Solution | Volume (uL) | Final Concentration | |
|---|---|---|---|
| HFE-7500 | 400 | — | |
| 008-FluoroSurfactant 20% | 100 | 2% | w/v |
| w/v in HFE-7500 | | | |
| TEMED | 2 | 0.4% | v/v |
| Total | 502 | | |

6) Turn on the microscope and place the microfluidic device on the microscope stage. 7) Assemble the fluidic circuit with 3 syringes connected to the 3 inlets using the tubing and the right-angle couplers. Add a right-angle couple to the outlet and attach tubing to direct the outflow to a 1.5 mL tube containing 150 μL of mineral oil. 8) Set the fluidic pressures to the following settings: a) Cell input: 1.2 psi; b) Lysis input: 1.4 psi; c) oil input 1.5 psi. 9) Open the pressure valves in the following order waiting one second before opening the next valve: Cell input, lysis input, and oil input. 10) After collection is complete, incubate the tube in 55 C for 30 minutes and then let the gel polymerize overnight at room temperature.

Droplet Breakage—The following protocol was used to effectuate breakage of the droplets at the appropriate timepoint. 1) Using a pipette, remove the upper mineral oil layer and the lower HFE-7500 layer; 2) Add 600 uL of 6×SSC and 150 uL of PFO and vortex the beads briefly to break the gel beads out of the emulsion on ice; 3) Centrifuge 300 g for 2 minutes at 4 C to pellet the beads and remove the top and bottom layers leaving the gel beads in the middle on ice; 4) Add another 5 mL of 6×SSC and remove the top and; 5) Wash once with 5×Reverse Transcription Buffer

cDNA Synthesis was performed according to the following protocol: 1) The reverse transcription reaction buffer was prepared according to the below formula.

| Reverse Transcription | | | |
|---|---|---|---|
| Reaction | Volume (uL) | Final Concentration | |
| 1X Reverse Transcription | 225 | 1X | |
| Buffer | | | |
| Maxima H (200 U/uL) | 37.5 | 10 | U/μL |
| 10 mM dNTPs | 37.5 | 0.5 | μM |

| | | |
|---|---|---|
| Encapsulated Beads | 450 | — |
| Total | 750 | |

2) Incubate under rotation for 30 minutes at room temperature and then incubate the reaction at 42 C for 60 minutes. 3) Finish the reaction by incubating tubes under rotation at 50 C for 60 minutes. 4) Add 750 uL of binding buffer and incubate for 5 minutes at room temperature to denature enzymes. Then add 1.5 uL of tween-20 and mix well to prevent beads from sticking to the edge of the tube. 5) Then wash twice with Tris-Tween buffer and twice with PBS (for secondary strand synthesis protocol) or tagmentation buffer (no DMF) (for hybrid Tn5 protocol). 6) Set up secondary strand synthesis reaction on ice (formula below)

| Secondary Strand | | |
|---|---|---|
| Synthesis Reaction | Volume (uL) | Final Concentration |
| Secondary Strand Synthesis | 80 | 1X |
| Buffer | | |
| Secondary Strand Synthesis | 40 | 1X |
| Enzyme Mix | | |
| Beads in PBS | 680 | — |
| Total | 800 | |

7) Incubate overnight at 16 C. 8) Add 750 μL of binding buffer and incubate 5 minutes at room temperature to denature enzymes. Then add 1.5 uL of tween-20 and mix well to prevent beads from sticking to the edge of the tube. 9) Then wash twice with Tris-tween buffer and wash twice with water.

Combinatorial Indexing was performed according to the following method: 1) Anneal transposons and mosaic end sequences by setting up the following reaction:

| Transposon Annealing | | Final |
|---|---|---|
| Reaction | Volume (uL) | Concentration |
| Transposon annealing buffer | 1 | 1X |
| Mosaic End (100 μM) | 1 | 10 μM |
| Indexed Transposon (100 μM) | 1 | 10 μM |
| H₂O | 7 | |
| Total | 10 | |

2) Anneal the transposons with the ramp down protocol:

| Transposon Annealing | | |
|---|---|---|
| Reaction | Temperature | Time |
| 1 | 95 C. | 5:00 |
| 2 | Cool to 65 C. | −0.1 C./s |
| 3 | 65 C. | 5:00 |
| 4 | Cool to 4 C. | −0.1 C./s |

3) Set up the transposase reaction:

| | Volume | Final | |
|---|---|---|---|
| Transposase Loading | (uL) | Concentration | |
| Annealed Transposons | 2 | 10 | μM |
| Unloaded Transposase (0.4 mg/mL) | 2 | 0.2 | mg/mL |
| Total | 4 | | |

4) Load the transposase for 30 minutes at 23 C in a thermocycler then add 2 μL of 100% Glycerol

5) Set up multiplexed tagmentation reaction and mix well adding the gel beads last:

| | Volume | Final |
|---|---|---|
| Tagmentation Reaction | (uL) | Concentration |
| Loaded indexed transposons | 2 | 0.0025 mg/mL |
| (0.025 mg/mL diluted in 1X | | |
| tagmentation buffer) | | |
| Encapsulated cells | 8 | — |
| (30 cells/uL) | | |
| 2X Tagmentation Buffer | 10 | 1X Tagmentation Buffer |
| Total | 20 | |

6) Incubate samples at 55 C shaking at 600 RPM for 90 minutes. Then add 200 μL of Tris-Tween buffer to stop the reaction. 7) Pool all reactions and pellet beads 300 g for 2 minutes. 8) Wash with Tris-Tween 20 buffer twice and then wash with H₂O twice. 9) Set up ligation multiplexed ligation reaction and mix well adding the gel beads last.

| | Volume | Final | |
|---|---|---|---|
| Ligation Reaction | (uL) | Concentration | |
| 2X T7 Ligation Buffer | 10 | 1X | |
| T7 Ligase (3 U/uL) | 2.5 | 0.375 | U/uL |
| Splint Oligo (100 μM) | 1.8 | 9 | μM |
| Indexed adapter (50 μM) | 1.2 | 3 | μM |

| | | |
|---|---|---|
| Tagmented Gel Beads | 4.5 | — |

10) Incubate at 25 C shaking at 600 RPM for 60 minutes and then heat inactivate the ligase with 65 C for 10 minutes. 11) Add 150 uL of Tris-Tween buffer and then pool all reactions. 12) Wash twice in Tris-Tween buffer and then wash twice in H₂O. 13) Adjust the bead concentration to 10 cells/uL. 14) Split into the final barcoding plate and denature the Tn5:

| | Volume | Final |
|---|---|---|
| Tn5 Denaturation | (uL) | Concentration |
| 0.3% SDS | 2 | 0.1% |
| Gel beads (10 cells/uL) | 4 | 6.6 cells/uL |
| Total | 6 | |

15) Vortex samples to mix well and incubate 55 C for 15 minutes and then add 1.5 uL of 10% Triton-X to quench the SDS. 16) Incubate samples for 55 C for 15 minutes and then set up gap filling reaction (change to 20 uL for RNA).

| | Volume | Final | |
|---|---|---|---|
| Tn5 Gapfilling Reaction | (uL) | Concentration | |
| 5X Q5 Buffer | 8 | 1X | |
| dNTPs replacing the dCTP | 1.3 | 0.25 | mM |
| with dmCTP (7.6 mM) | | | |
| Q5 Polymerase (2 U/uL) | 0.4 | 0.2 | U/uL |
| RNA Linear Amplification | 2 | 0.5 | μM |

| | |
|---|---|
| Primer (10 μM) | |
| SDS quenched samples | 7.5 |
| H₂O | 22.8 |
| Total | 40 |

17) Incubate 72 C for 10 minutes to run the gap filling reaction. 18) Linearly amplify the RNA for 10 cycles to extract out the RNA from the gel beads.

| Step | Temperature | Time |
|---|---|---|
| 1 | 72 C. | 10:00 |
| 2 | 98 C. | 3:00 |
| 3 | 98 C. | 0:20 |
| 4 | 59 C. | 1:00 |
| 5 | 72 C. | 2:00 |
| 6 | GOTO Step 3 × 9 | |

19) Pellet beads and carefully take 20 uL of the supernatant of the PCR reaction containing the cDNA. 20) Add 1 μL of indexed i5 primer and exponentially amplify the RNA for 7 cycles.

| Step | Temperature | Time |
|---|---|---|
| 1 | 98 C. | 0:10 |
| 2 | 59 C. | 0:30 |
| 3 | 72 C. | 1:00 |
| 4 | GOTO Step 1 × 6 | |

21) Follow the EZ-96 DNA Methylation-Direct MagPrep instructions and add 130 uL of bisuflite conversion reagent to the remaining 20 uL of the gap filled reaction to bisulfite convert the DNA library. 22) Perform bisulfite conversion and desulphonation according to the instructions on EZ-96 DNA Methylation-Direct MagPrep. During the elution step, add 20 uL of H₂O and mix well. 23) Take the whole volume including the magnetic beads and transfer each reaction to a new 96 well plate.

Post Bisulfite Conversion Processing was performed according to the following protocol 1) Set up the final barcoding linear amplification for the methylated DNA library.

| | Volume | Final |
|---|---|---|
| Linear Barcoding Reaction | (uL) | Concentration |
| 2X KAPA HiFi U + Master Mix | 25 | 1X |
| 10 μm indexed P5 primer | 2.5 | 0.5 μM |
| Bisulfite Converted Beads | 20 | — |
| Total | 50 | |

2) Perform 20 cycles of linear amplification with the following PCR conditions.

| Step | Temperature | Time |
|---|---|---|
| 1 | 98 C. | 3:00 |
| 2 | 98 C. | 0:20 |
| 3 | 59 C. | 1:00 |
| 4 | 72 C. | 2:00 |
| 5 | GOTO Step 2 × 19 | |

3) Add 2.5 uL of rSAP and incubate all samples at 37 C for 30 minutes and heat inactivate 65 C for 5 minutes. 4) Using a magnetic rack, remove the supernatant from the PCR reactions into a new PCR plate. 5) Perform 1.2×bead purification on all samples and elute 10 uL in a new 96 well plate. 6) To set up the adaptase reaction, first incubate the plate at 95 C for 3 minutes and then immediately place the plate on ice for 2 minutes. 7) As quickly as possible, set up the adaptase reaction for all samples by adding the following:

| | Volume | Final |
|---|---|---|
| Adaptase Reaction | (uL) | Concentration |
| Buffer G1 | 2 | — |
| Reagent G2 | 2 | — |
| Reagent G3 | 1.25 | — |
| Enzyme G4 | 0.5 | — |
| Enzyme G5 | 0.5 | — |
| Low EDTA TE | 4.25 | — |
| Bead purified samples | 10 | — |
| Total | 20.5 | |

8) Run the adaptase reaction conditions by first incubating all samples at 37 C for 30 minutes followed by heat denaturation of the enzyme by incubating the samples at 95 C for 2 minutes. 9) Set up the final qPCR reaction as per the following:

| | Volume | Final | |
|---|---|---|---|
| Final DNA PCR | (uL) | Concentration | |
| 2X KAPA HiFi Master Mix | 25 | 1X | |
| 10 μM P5 primer | 3 | 0.6 | μM |
| 10 μM DNA i7 primer | 5 | 1 | μM |

| | | |
|---|---|---|
| Adaptase reaction | 20 | — |
| SYBR Green 100X | 0.2 | 1X |
| Total | 50 | |

10) Amplify with the following PCR conditions (fill in details later) for 10 cycles per the below:

| Step | Temperature | Time |
|---|---|---|
| 1 | 98 C. | 3:00 |
| 2 | 98 C. | 0:10 |
| 3 | 59 C. | 0:30 |
| 4 | 72 C. | 1:00 |
| 5 | GOTO Step 2 × 9 | |

11) Bead purify with 0.8×SPRI bead ratio the individually exponentially amplified RNA library and set up the final indexed i7 PCR per the below:

| | Volume | Final | |
|---|---|---|---|
| Final RNA PCR | (uL) | Concentration | |
| 2X KAPA HiFi Master Mix | 25 | 1X | |
| 10 μM P5 primer | 2.5 | 0.6 | μM |
| 10 μM RNA i7 primer | 2.5 | 1 | μM |

| | | |
|---|---|---|
| RNA exponentially amplified reaction | 20 | — |
| SYBR Green 100X | 0.5 | 1X |
| Total | 50 | |

12) Similarly, bead purify the DNA library with 0.8×SPRI bead ratio individually and set up the final PCR per the below:

| | Volume | Final | |
|---|---|---|---|
| Final RNA PCR | (uL) | Concentration | |
| 2X KAPA HiFi Master Mix | 25 | 1X | |
| 10 μM P5 primer | 2.5 | 0.6 | μM |
| 10 μM P7 primer | 2.5 | 1 | μM |

| | | |
|---|---|---|
| RNA exponentially amplified reaction | 20 | — |
| SYBR Green 100X | 0.5 | 1X |
| Total | 50 | |

13) Amplify with the following PCR conditions for ˜12 cycles right before PCR saturation per the below:

| Step | Temperature | Time |
|---|---|---|
| 1 | 98 C. | 3:00 |
| 2 | 98 C. | 0:10 |
| 3 | 59 C. | 0:30 |
| 4 | 72 C. | 1:00 |
| 5 | GOTO Step 2 × 12 | |

14) Pool the individual DNA and RNA reactions separately and perform 2 0.8×SPRI bead ratio purifications to clean up the library for sequencing. 15) Methylated DNA libraries were sequenced 130 cycles read 1, 10 cycles index 1, 37 cycles index 2, and 100 cycles read 2. Typically, this library was sequenced to a depth of 500,000 reads/cell. 16) RNA libraries were sequenced 130 cycles read 1, 10 cycles index 1, 37 cycles index 2, and 40 cycles read 2. Typically, this library was sequenced to a depth of 10,000 reads/cell.

### Bioinformatic Methods

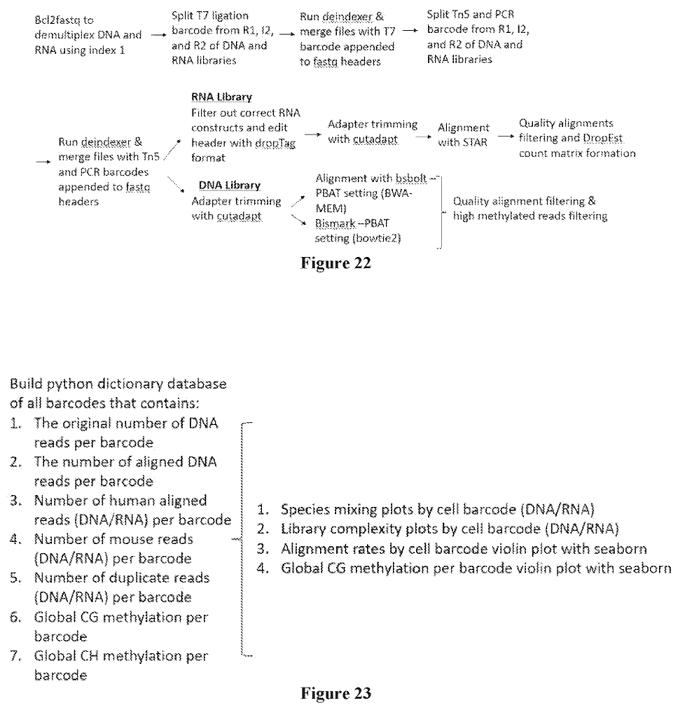

Pre-Processing-Libraries were first demultiplexed using index 1 used to distinguish RNA libraries from DNA ones using bcl2fastq. The ligation barcode located in the last 10 bases of the index 2 read was then extracted. Configuration files and barcode lists were assembled according to the formatting required by deindexer. Deindexer was then used to demultiplex the DNA reads and RNA reads by the ligation barcode. In addition, the index 2 read was demultiplexed by deindexer. Both the DNA and RNA reads were then concatenated into a single file but keeping the read ID of each read was edited to the following notation: @xx, where xx is the ligation barcode number that the read was demultiplexed with. The Tn5 barcode located in the first 10 bases of read 1 were then extracted followed by the PCR barcode located in the last 10 bases of index 2 for both the DNA and RNA libraries. Deindexer was then used to demultiplex the DNA reads and RNA reads by both the Tn5 barcode and PCR barcode. Both the DNA and RNA reads were then concatenated into a single file but keeping the read ID of each read was edited to the following notation: @xx.yy.zz, where xx is the ligation barcode number, yy is the Tn5 barcode, and zz is the PCR barcode. The RNA library was then filtered for the correct construct by looking for a “TTTT” sequence in the 32-36 positions in read 2. In addition, the UMI was extracted from the positions 23-30 in read 2 and the read ID of read 1 was edited to the format: @!xx.yy zz #UMI. This read ID matches the format required for downstream analyses using the dropEst package. Both the read 1 DNA and RNA libraries were then trimmed for the Tn5 adapter, adaptase adapter, and polyT sequences using cutadapt. An additional 10 bases from the DNA library are trimmed as this is artificially methylated during the gap filling steps. The DNA reads were mapped with the bsbolt package which is a BWA-MEM wrapper for bisulfite converted sequence mapping using the PBAT. In addition, the DNA reads were mapped with bismark which is a bowtie2 wrapper for bisulfite converted sequence mapping using the PBAT settings. The RNA reads were mapped with STAR. Both DNA and RNA libraries are filtered for high quality reads. The RNA reads were then input into the dropEst package which performs UMI collapse and creates a counts matrix for secondary analysis. The highly methylated reads in the DNA libraries were removed using a G to A conversion cutoff to remove cDNA reads that are artificially methylated prior to bisulfite conversion. The duplicate reads in the DNA library were then removed. FIG. 22 illustrates the pre-processing pipeline described herein.

Primary Analysis Pipeline—A python dictionary was created to organize all the important sequencing information by cell barcode. The global CG and CH methylation information is encoded in the bismark alignment files under the field “XM”. Methylated CH is encoded as H while unmethylated CH is encoded as h. Similarly, methylated CG is encoded as Z while unmethylated CG is encoded as z. These encodings were quantified per cell barcode in the creation of the database. FIG. 23 summarizes the database structure

Secondary Analysis Pipeline—The RNA alignment files were first coordinate sorted and duplicate reads were removed. The htseq software was used to create an RNA gene x sample counts matrix using htseq-count. This counts matrix contained the bulked RNA counts of encapsulated HCT116, RNA counts from an HCT116 in-tube control, and RNA counts from a U87 in-tube control all created by the RNA-seq protocol. The analysis was performed at the bulk level to increase gene coverage. The counts matrices were then input into scanpy where the counts were log normalized and converted to counts per million. The log normalized RNA counts of each sample pair-wise were plotted and marker genes obtained from literature of each cell type were labeled. At the single cell level, the dropEst counts matrix was input into Seurat. Using Seurat, barcodes were filtered with gene counts <200 and >1000 (potential doublets). The counts matrix was then similarly log normalized. Further analysis such as clustering and cell type identification follows previously published methods using Seurat.

### REFERENCES

Ahn, Jongseong, Sunghoon Heo, Jihyun Lee, and Duhee Bang. 2021. “Introduction to Single-Cell Dna Methylation Profiling Methods.” Biomolecules 11 (7). doi.org/10.3390/biom11071013.
Angermueller, Christof, Stephen J. Clark, Heather J. Lee, Iain C. Macaulay, Mabel J. Teng, Tim Xiaoming Hu, Felix Krueger, et al. 2016. “Parallel Single-Cell Sequencing Links Transcriptional and Epigenetic Heterogeneity.” Nature Methods 13 (3): 229-32. doi.org/10.1038/nmeth.3728.
Argelaguet, Ricard, Stephen J. Clark, Hisham Mohammed, L. Carine Stapel, Christel Krueger, Chantriolnt Andreas Kapourani, Ivan Imaz-Rosshandler, et al. 2019. “Multi-Omics Profiling of Mouse Gastrulation at Single-Cell Resolution.” Nature 576 (7787): 487-91. doi.org/10.1038/s41586-019-1825-8.
Argelaguet, Ricard, Britta Velten, Damien Arnol, Sascha Dietrich, Thorsten Zenz, John C Marioni, Florian Buettner, Wolfgang Huber, and Oliver Stegle. 2018. “Multi-Omics Factor Analysis—a Framework for Unsupervised Integration of Multi-omics Data Sets.” Molecular Systems Biology 14 (6). doi.org/10.15252/msb.20178124.
Callaway, Edward M., Hong-Wei Dong, Joseph R. Ecker, Michael J. Hawrylycz, Z. Josh Huang, Ed S. Lein, John Ngai, et al. 2021. “A Multimodal Cell Census and Atlas of the Mammalian Primary Motor Cortex.” Nature 598 (7879): 86-102. doi.org/10.1038/s41586-021-03950-0.
Cao, Junyue, Diana R O′day, Hannah A Pliner, Paul D Kingsley, Mei Deng, Riza M Daza, Michael A Zager, et al. 2020. “A Human Cell Atlas of Fetal Gene Expression Techniques and Performed Sci-RNA-Seq3 Experiments with Assistance from R HHS Public Access.” Science 370 (6518). doi.org/10.17504/protocols.io.9yih 7ue.
Cao, Junyue, Malte Spielmann, Xiaojie Qiu, Xingfan Huang, Daniel M. Ibrahim, Andrew J. Hill, Fan Zhang, et al. 2019. “The Single-Cell Transcriptional Landscape of Mammalian Organogenesis.” Nature 566 (7745): 496-502. doi.org/10.1038/s41586-019-0969-x.
Chen, Song, Blue B. Lake, and Kun Zhang. 2019. “High-Throughput Sequencing of the Transcriptome and Chromatin Accessibility in the Same Cell.” Nature Biotechnology 37 (12): 1452-57. doi.org/10.1038/s41587-019-0290-0.
Clark, Stephen J., Ricard Argelaguet, Chantriolnt Andreas Kapourani, Thomas M. Stubbs, Heather J. Lee, Celia Alda-Catalinas, Felix Krueger, et al. 2018. “ScNMT-Seq Enables Joint Profiling of Chromatin Accessibility DNA Methylation and Transcription in Single Cells e.” Nature Communications 9 (1). doi.org/10.1038/s41467-018-03149-4.
Domcke, Silvia, Andrew J. Hill, Riza M. Daza, Junyue Cao, Diana R. O'Day, Hannah A. Pliner, Kimberly A. Aldinger, Dmitry Pokholok, Fan Zhang, Jennifer H. Milbank, Michael A. Zager, Ian A. Glass, Frank J. Steemers, Dan Doherty, Cole Trapnel, et al. 2020. “A Human Cell Atlas of Fetal Chromatin Accessibility.” Science 370 (6518). doi.org/10.1126/science.aba7612.
Domcke, Silvia, Andrew J. Hill, Riza M. Daza, Junyue Cao, Diana R. O'Day, Hannah A. Pliner, Kimberly A. Aldinger, Dmitry Pokholok, Fan Zhang, Jennifer H. Milbank, Michael A. Zager, Ian A. Glass, Frank J. Steemers, Dan Doherty, Cole Trapnell, et al. 2020. “A Human Cell Atlas of Fetal Chromatin Accessibility.” Science 370 (6518). doi.org/10.1126/science.aba7612.
Dzieran, Johanna, Aida Rodriguez Garcia, Ulrica Kristina Westermark, Aine Brigette Henley, Elena Eyre Sánchez, Catarina Träger, Henrik Johan Johansson, Janne Lehtio, and Marie Arsenian-Henriksson. 2018. “MYCN-Amplified Neuroblastoma Maintains an Aggressive and Undifferentiated Phenotype by Deregulation of Estrogen and NGF Signaling.” Proceedings of the National Academy of Sciences of the United States of America 115 (6): F1229-38. doi.org/10.1073/pnas. 1710901115.
Gu, Hongcang, Ayush T. Raman, Xiaoxue Wang, Federico Gaiti, Ronan Chaligne, Arman W. Mohammad, Aleksandra Arczewska, et al. 2021. “Smart-RRBS for Single-Cell Methylome and Transcriptome Analysis.” Nature Protocols. Nature Research. doi.org/10.1038/s41596-021-00571-9.
Heard, Edith, Philippe Clerc, and Philip Avner. 1997. “X-CHROMOSOME INACTIVATION IN MAMMALS.” www.annualreviews.org.
Hu, Youjin, Kevin Huang, Qin An, Guizhen Du, Ganlu Hu, Jinfeng Xue, Xianmin Zhu, Cun Yu Wang, Zhigang Xue, and Guoping Fan. 2016. “Simultaneous Profiling of Transcriptome and DNA Methylome from a Single Cell.” Genome Biology 17 (1). doi.org/10.1186/s13059-016-0950-z.
Klein, Allon M., Linas Mazutis, Ilke Akartuna, Naren Tallapragada, Adrian Veres, Victor Li, Leonid Peshkin, David A. Weitz, and Marc W. Kirschner. 2015. “Droplet Barcoding for Single-Cell Transcriptomics Applied to Embryonic Stem Cells.” Cell 161 (5): 1187-1201. doi.org/10.1016/j.cell.2015.04.044.
Kriaucionis, Skirmantas, and Nathaniel Heintz. n.d. “The Nuclear DNA Base, 5-Hydroxymethylcytosine Is Present in Brain and Enriched in Purkinje Neurons.”
Lake, Blue B., Song Chen, Brandon C. Sos, Jean Fan, Gwendolyn E. Kaeser, Yun C. Yung, Thu E. Duong, et al. 2018. “Integrative Single-Cell Analysis of Transcriptional and Epigenetic States in the Human Adult Brain.” Nature Biotechnology 36 (1): 70-80. doi.org/10.1038/nbt.4038.
Lake, Blue B, Rajasree Menon, Seth Winfree, Qiwen Hu, Ricardo Melo Ferreira, Daria Barwinska, Edgar A Otto, et al. n.d. “An Atlas of Healthy and Injured Cell 1 States and Niches in the Human Kidney.” doi.org/10.1101/2021.07.28.454201.
Lan, Freeman, Benjamin Demaree, Noorsher Ahmed, and Adam R. Abate. 2017. “Single-Cell Genome Sequencing at Ultra-High-Throughput with Microfluidic Droplet Barcoding.” Nature Biotechnology 35 (7): 640-46. doi.org/10.1038/nbt.3880.
Lengauer, Christoph, Kenneth W Kinzler, and Bert Vogelstein. 1997. “DNA Methylation and Genetic Instability in Colorectal Cancer Cells.” Medical Sciences. Vol. 94. www.pnas.org.
Li, Siran, Jude Kendall, Sarah Park, Zihua Wang, Joan Alexander, Andrea Moffitt, Nissim Ranade, et al. 2020. “Copolymerization of Single-Cell Nucleic Acids into Balls of Acrylamide Gel.” Genome Research 30 (1): 49-61. doi.org/10.1101/gr.253047.119.
Liu, Jialin, Chao Gao, Joshua Sodicoff, Velina Kozareva, Evan Z. Macosko, and Joshua D. Welch. 2020. “Jointly Defining Cell Types from Multiple Single-Cell Datasets Using LIGER.” Nature Protocols 15 (11): 3632-62. doi.org/10.1038/s41596-020-0391-8.
Luo, Chongyuan, Hanging Liu, Fangming Xie, Ethan J. Armand, Kimberly Siletti, Trygve E. Bakken, Rongxin Fang, et al. 2022. “Single Nucleus Multi-Omics Identifies Human Cortical Cell Regulatory Genome Diversity.” Cell Genomics 2 (3): 100107. doi.org/10.1016/j.xgen.2022.100107.
Luo, Chongyuan, Angeline Rivkin, Jingtian Zhou, Justin P. Sandoval, Laurie Kurihara, Jacinta Lucero, Rosa Castanon, et al. 2018. “Robust Single-Cell DNA Methylome Profiling with SnmC-Seq2.” Nature Communications 9 (1). doi.org/10.1038/s41467-018-06355-2.
M. Dunn, Christopher, Michael C. Nevitt, John A. Lynch, and Matlock A. Jeffries. 2019. “A Pilot Study of Peripheral Blood DNA Methylation Models as Predictors of Knee Osteoarthritis Radiographic Progression: Data from the Osteoarthritis Initiative (OAI).” Scientific Reports 9 (1). doi.org/10.1038/s41598-019-53298-9
Macosko, Evan Z., Anindita Basu, Rahul Satija, James Nemesh, Karthik Shekhar, Melissa Goldman, Itay Tirosh, et al. 2015. “Highly Parallel Genome-Wide Expression Profiling of Individual Cells Using Nanoliter Droplets.” Cell 161 (5): 1202-14. doi.org/10.1016/j.cell.2015.05.002.
Mitra, Robi D, and George M Church. 1999. “In Situ Localized Amplification and Contact Replication of Many Individual DNA Molecules.” Nucleic Acids Research. Vol. 27.
Mulqueen, Ryan M., Dmitry Pokholok, Steven J. Norberg, Kristof A. Torkenczy, Andrew J. Fields, Duanchen Sun, John R. Sinnamon, et al. 2018. “Highly Scalable Generation of DNA Methylation Profiles in Single Cells.” Nature Biotechnology 36 (5): 428-31. doi.org/10.1038/nbt.4112.
Mulqueen, Ryan M., Dmitry Pokholok, Brendan L. O'Connell, Casey A. Thornton, Fan Zhang, Brian
J. O'Roak, Jason Link, et al. 2021. “High-Content Single-Cell Combinatorial Indexing.” Nature Biotechnology 39 (12): 1574-80. doi.org/10.1038/s41587-021-00962-z.
Picelli, Simone, Åsa K. Björklund, Björn Reinius, Sven Sagasser, Gösta Winberg, and Rickard Sandberg. 2014. “Tn5 Transposase and Tagmentation Procedures for Massively Scaled Sequencing Projects.” Genome Research 24 (12): 2033-40. doi.org/10.1101/gr.177881.114.
Picelli, Simone, Omid R. Faridani, Åsa K. Björklund, Gösta Winberg, Sven Sagasser, and Rickard Sandberg. 2014. “Full-Length RNA-Seq from Single Cells Using Smart-Seq2.” Nature Protocols 9 (1): 171-81. doi.org/10.1038/nprot.2014.006.
Pliner, Hannah A., Jonathan S. Packer, José L. McFaline-Figueroa, Darren A. Cusanovich, Riza M. Daza, Delasa Aghamirzaie, Sanjay Srivatsan, et al. 2018. “Cicero Predicts Cis-Regulatory DNA Interactions from Single-Cell Chromatin Accessibility Data.” Molecular Cell 71 (5): 858-871.e8. doi.org/10.1016/j.molcel.2018.06.044.
Plongthongkum, Nongluk, Dinh Diep, Song Chen, Blue B. Lake, and Kun Zhang. 2021. “Scalable Dual-Omics Profiling with Single-Nucleus Chromatin Accessibility and MRNA Expression Sequencing 2 (SNARE-Seq2).” Nature Protocols 16 (11): 4992-5029. doi.org/10.1038/s41596-021-00507-3.
Pluen, Alain, Paolo A Netti, Rakesh K Jain, and David A Berk. 1999. “Diffusion of Macromolecules in Agarose Gels: Comparison of Linear and Globular Configurations.”
Quake, Stephen R. 2022. “A Decade of Molecular Cell Atlases.” Trends in Genetics. Elsevier Ltd. doi.org/10.1016/j.tig.2022.01.004.
Rosenberg, Alexander B, † Charles, M Roco, Richard A Muscat, Anna Kuchina, Paul Sample, Zizhen Yao, et al. n.d. “Single-Cell Profiling of the Developing Mouse Brain and Spinal Cord with Split-Pool Barcoding.” www.science.org.
Sharifi-Zarchi, Ali, Daniela Gerovska, Kenjiro Adachi, Mehdi Totonchi, Hamid Pezeshk, Ryan J. Taft, Hans R. Schöler, et al. 2017. “DNA Methylation Regulates Discrimination of Enhancers from Promoters through a H3K4me1-H3K4me3 Seesaw Mechanism.” BMC Genomics 18 (1). hdoi.org/10.1186/s12864-017-4353-7.
Travaglini, Kyle J., Ahmad N. Nabhan, Lolita Penland, Rahul Sinha, Astrid Gillich, Rene v. Sit, Stephen Chang, et al. 2020. “A Molecular Cell Atlas of the Human Lung from Single-Cell RNA Sequencing.” Nature 587 (7835): 619-25. doi.org/10.1038/s41586-020-2922-4.
Uzun, Yasin, Hao Wu, and Kai Tan. 2021. “Predictive Modeling of Single-Cell DNA Methylome Data Enhances Integration with Transcriptome Data.” Genome Research 31 (1): 101-9. doi.org/10.1101/gr.267047.120.
Wu, Douglas C., and Alan M. Lambowitz. 2017. “Facile Single-Stranded DNA Sequencing of Human Plasma DNA via Thermostable Group II Intron Reverse Transcriptase Template Switching.” Scientific Reports 7 (1). doi.org/10.1038/s41598-017-09064-w.
Xu, Liyi, Ilana L. Brito, Eric J. Alm, and Paul C. Blainey. 2016. “Virtual Microfluidics for Digital Quantification and Single-Cell Sequencing.” Nature Methods 13 (9): 759-62. doi.org/10.1038/nmeth.3955.
Zhu, Chenxu, Miao Yu, Hui Huang, Ivan Juric, Armen Abnousi, Rong Hu, Jacinta Lucero, M. Margarita Behrens, Ming Hu, and Bing Ren. 2019. “An Ultra High-Throughput Method for Single-Cell Joint Analysis of Open Chromatin and Transcriptome.” Nature Structural and Molecular Biology 26 (11): 1063-70. doi.org/10.1038/s41594-019-0323-x.
Zhu, Honglin, Chengsong Zhu, Wentao Mi, Tao Chen, Hongjun Zhao, Xiaoxia Zuo, Hui Luo, and Quan Zhen Li 2018 “Integration of Genome-Wide DNA Methylation and Transcription Uncovered Aberrant Methylation-Regulated Genes and Pathways in the Peripheral Blood Mononuclear Cells of Systemic Sclerosis.” International Journal of Rheumatology 2018. doi.org/10.1155/2018/7342472.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

What is claimed is:

1. A method of parallel single-cell sequencing, comprising:
a) providing a plurality of cell nuclei or lysate thereof encapsulated in gel beads;
b) performing reverse transcription within the gel beads to form complementary DNA (cDNA);
c) partitioning the gel beads to a first plurality of vessels and adding a first DNA barcode to the cDNA and genomic DNA within the gel beads, each of the vessels of the first plurality of vessels having a unique first DNA barcode sequence;
d) pooling and re-partitioning the gel beads to a second plurality of vessels and adding a second DNA barcode to the cDNA and genomic DNA within the gel beads, each of the vessels of the second plurality of vessels having a unique second DNA barcode sequence;
e) pooling and performing a second re-partitioning of the gel beads to a third plurality of vessels;
f) separating the cDNA from the genomic DNA;
g) adding a third DNA barcode to the separated cDNA;
h) performing bisulfite conversion of the separated genomic DNA and adding a third DNA barcode to the separated genomic DNA, wherein the third DNA barcode sequence is the same for genomic DNA and cDNA derived from the same cell nucleus; and
i) sequencing the cDNA and the genomic DNA.

2. The method of claim 1, wherein individual gel beads comprise a single cell nucleus or lysate thereof.

3. The method of claim 1, wherein providing the plurality of cell nuclei or lysate thereof encapsulated in gel beads comprises encapsulating the cell nuclei with a lysis buffer within a polymer matrix, wherein the polymer matrix forms the gel beads.

4. The method of claim 3, wherein the encapsulating comprises mixing the cell nuclei, the lysis buffer, and the polymer matrix within a water-in-oil droplet.

5. The method of claim 1, wherein the gel beads are comprised of an acrylamide polymer.

6. The method of claim 5, wherein the acrylamide polymer is prepared from acrylamide and bis-acrylamide in a ratio of about 100:1 (w/w).

7. The method of claim 1, wherein the gel beads have an average diameter of from about 100 to about 150 microns.

8. The method of claim 1, wherein the gel beads comprise mRNA capture probes covalently attached to the gel beads.

9. The method of claim 8, wherein the mRNA capture probes act as reverse transcription primers during the reverse transcription step.

10. The method of claim 1, wherein adding the first DNA barcode to the cDNA and the genomic DNA comprises transposon barcoding.

11. The method of claim 10, wherein the transposon barcoding is performed with transposon Tn5.

12. The method of claim 1, wherein the second DNA barcode is added to the cDNA and the genomic DNA by ligation.

13. The method of claim 12, wherein the ligation is performed with a T7 ligase.

14. The method of claim 1, further comprising amplifying the cDNA within the gel beads within the third plurality of vessels.

15. The method of claim 1, wherein separating the cDNA from the genomic DNA comprises centrifuging the gel beads to form a pellet and removing supernatant containing the cDNA.

16. The method of claim 15, wherein the third DNA barcode is added to the cDNA by polymerase chain reaction (PCR) of the cDNA in the supernatant.

17. The method of claim 15, wherein the performing bisulfite conversion of the separated genomic DNA comprises adding bisulfite conversion reagents to the pellet.

18. The method of claim 1, wherein the third DNA barcode is added to the genomic DNA by PCR of the genomic DNA.

19. The method of claim 1, further comprising a gap filling step of amplifying the nucleic acids in the presence of a 5-methylcytosine dNTP.

20. The method of claim 1, wherein the method obtains single cell sequencing data from at least 10,000 cell nuclei.

21. The method of claim 1, wherein the method obtains single cell sequencing data from at least 100,000 cell nuclei.

22. The method of claim 1, wherein each of the first, second, and third plurality of vessels comprises at least 96 individual vessels.

23. The method of claim 1, wherein each individual vessel of the first plurality of vessels comprises at least 200 gel beads containing a cell nucleus.
